(19) United States
(12) Patent Application Publication
(10) Pub. No.: US 2026/0055241 A1
(43) Pub. Date: Feb. 26, 2026

(54) STORAGE AND RELEASE OF BIOMOLECULES

(71) Applicants: Cache DNA, Inc., San Carlos, CA (US); Massachusetts Institute of Technology, Cambridge, MA (US)

(72) Inventors: Elisabeth PRINCE, Cambridge, MA (US); Ho Fung CHENG, Cambridge, MA (US); James L. BANAL, San Carlos, CA (US); Jeremiah A. JOHNSON, Cambridge, MA (US)

(21) Appl. No.: 19/306,283

(22) Filed: Aug. 21, 2025

Related U.S. Application Data
(63) Continuation of application No. PCT/US2023/085375, filed on Dec. 21, 2023.
(60) Provisional application No. 63/447,843, filed on Feb. 23, 2023.
(60) Provisional application No. 63/501,628, filed on May 11, 2023.

(51) Int. Cl. C08J 3/00; C07C 69/54; C07C 229/30; C08G 63/66

(52) U.S. Cl. CPC C08J 3/005; C07C 69/54; C07C 229/30; C08G 63/66

(57) ABSTRACT

Provided herein are compositions, methods, and kits relating to the formation and deconstruction of thermosets capable of storing biomolecules.

### CROSS-REFERENCE

This application is a continuation of PCT/US2023/085375 filed Dec. 21, 2023, which claims priority to U.S. Provisional Ser. No. 63/501,628, filed May 11, 2023, and U.S. Provisional Patent Application, 63/447,843, filed Feb. 23, 2023, each of which is incorporated by reference herein in its entirety for all purposes.

### STATEMENT AS TO FEDERALLY SPONSORED RESEARCH

This invention was made with government support under 2136447 awarded by the National Science Foundation (NSF). The government has certain rights in the invention.

### BACKGROUND

The central dogma of biology proceeds from DNA to RNA then proteins. These biomolecules play critical roles in sustaining life: DNA encodes the information for protein synthesis while RNA carries out the instruction encoded on the DNA. Proteins carry out most biological processes. The explosion and advances of omics technologies have driven the demand to understand individuals'health and predisposition to diseases through the collection, storage, and analyses of DNA, RNA, and proteins. Omics technologies that analyze nucleic acids, e.g., genomics and transcriptomics, are now scientifically advanced and commercialized at scale.

### SUMMARY

The present disclosure provides methods and systems for alternative encapsulation chemistry using hydrophobic polymers or polymer networks to realize a room-temperature storage and retrieval approach for biomolecules, e.g., DNA, RNA, or proteins, using hydrophobic biomolecules. Methods and systems of the present disclosure may be used for storage and retrieval of synthetic and naturally derived biomolecules. In some embodiments, biomolecules are combined with a compatibilizer, e.g., cationic amphiphilic polymers, and dispersed into a solution comprising monomers and crosslinkers that comprise the hydrophobic polymers or polymer networks. In some embodiments, biomolecules are bound to a solid support and suspended in a solution comprising monomers and crosslinkers that comprise the hydrophobic polymers or polymer networks. In some embodiments, biomolecules and encapsulation reagents are introduced into wells in a microplate comprising adsorbent particles using an automated liquid handling device. In some embodiments, biomolecules are trapped in emulsions using microfluidic channels controlled using electricity or photons, and encapsulated within the emulsion. In some embodiments, biomolecules and barcodes are combined and encased in emulsions comprising multiple layers of aqueous and organic solvents using microfluidic approaches. In some embodiments, permanent encapsulation using organic or inorganic polymers and barcoding proceeds in one operation. In some embodiments, biomolecules and barcodes are combined with magnetic or photonic elements to endow magnetic or photonic properties to the encapsulant. In some embodiments, molecular barcodes comprise non-standard nucleotides or non-phosphate backbones to improve the stability of the barcodes. In some embodiments, molecular barcodes can be attached using chemical synthesis or enzymes. In some embodiments, the hydrophobic network is diffusible. In some embodiments, selection of encapsulated samples proceeds by hybridization of probes that are complementary to the barcodes of interest. Probes may contain optical, chemical, or biochemical markers for optical or mechanical sorting using millifluidic or microfluidic approaches. In some embodiments, chemical and biochemical reactions can be performed on the tags to increase sorting throughput.

The present disclosure provides compositions comprising (a) 2-(dimethylamino)ethyl methacrylate (DMAEMA), (b) oligo(ethylene glycol) methyl ether methacrylate (OEGMA), and (c) styrene. The present disclosure also provides compositions comprising (a) [2-(acryloyloxy)ethyl]trimethylammonium chloride (AETAC), (b) oligo(ethylene glycol) methyl ether acrylate (OEGA), and (c) dodecyl acrylate (DDA).

The present disclosure provides compositions (a) comprising an amphiphilic polymer that forms a complex with a sequence-controlled polymer; (b) a hydrophilic agent; and (c) a hydrophobic agent. In some embodiments, the amphiphilic polymer is an amine-containing copolymer. In some embodiments, the amine-containing copolymer is DMAEMA. In some embodiments, the amphiphilic polymer is a cation-containing copolymer. In some embodiments, the cation-containing copolymer is AETAC. In some embodiments, the hydrophilic agent is OEGMA. In some embodiments, the hydrophilic agent is OEGA. In some embodiments, the hydrophobic agent is a hydrophobic monomer. In some embodiments, the hydrophobic agent is soluble in an organic solvent. In some embodiments, the hydrophobic agent is styrene. In some embodiments, the hydrophobic agent is DDA. In some embodiments, the ratio of a):b) is 1:1. In some embodiments, the ratio of a):c) is 1:1. In some embodiments, the ratio of a):c) is 4:1. In some embodiments, the ratio of a):b) is 6:94. In some embodiments, the ratio of a):b):c) is 1:1:1. In some embodiments, the ratio of a):b):c) is 6:74:20. In some embodiments, the ratio of a):b):c) is 6:62:31. In some embodiments, the ratio of a):b):c) is 6:54:40. In some embodiments, the ratio of a):b):c) is 6:31:63. In some embodiments, the composition further comprises a hydrophobic polymer or polymer network comprising a), b), and c). In some embodiments, the composition further comprises a non-aqueous solution comprising the hydrophobic polymer or polymer network. In some embodiments, the hydrophobic polymer or polymer network forms between 25° C. and 100° C. In some embodiments, the hydrophobic polymer or polymer network forms after application of light. In some embodiments, the hydrophobic polymer or polymer network is stable between 25° C. and 100° C. In some embodiments, the hydrophobic polymer or polymer network is stable for at least five years. In some embodiments, the hydrophobic polymer or polymer network is stable for at least ten years. In some embodiments, the hydrophobic polymer or polymer network is stable for at least 100 years. In some embodiments, the hydrophobic polymer or polymer network is not porous. In some embodiments, the hydrophobic polymer or polymer network is deconstructable upon application of a stimulus. In some embodiments, the stimulus comprises light. In some embodiments, the stimulus comprises heat. In some embodiments, the stimulus comprises a deconstruction solution. In some embodiments, the deconstruction solution comprises a nucleophile, an organic base, or a polar organic solvent. In some embodiments, the nucleophile is cysteamine. In some embodiments, the organic base is 1,8-diazabicyclo[5.4.0]undec-7-ene (DBU) or propylamine. In some embodiments, the polar organic solvent is aprotic. In some embodiments, the polar organic solvent is an organic base. In some embodiments, the polar organic solvent is dimethylformamide (DMF) or propylamine. In some embodiments, the deconstruction solution comprises cysteamine, DBU, and DMF. In some embodiments, the hydrophobic polymer or polymer network is deconstructable upon application of the stimulus for at most 5 hours. In some embodiments, the hydrophobic polymer or polymer network is deconstructable upon application of the stimulus for at most 2 hours. In some embodiments, the hydrophobic polymer or polymer network is deconstructable upon application of the stimulus for at most 1 hour. In some embodiments, the composition further comprises a sequence-controlled polymer, wherein the composition coats the sequence-controlled polymer. In some embodiments, the composition further comprises a hydrophobic polymer or polymer network comprising a), b), and c) that coats the sequence-controlled polymer. In some embodiments, the sequence-controlled polymer is a nucleic acid molecule. In some embodiments, the nucleic acid molecule is DNA. In some embodiments, the nucleic acid molecule is RNA. In some embodiments, the sequence-controlled polymer is a peptide. In some embodiments, the sequence-controlled polymer is an enzyme. In some embodiments, the sequence-controlled polymer is a protein. In some embodiments, a barcode or a tag is attached to the coated sequence-controlled polymer. In some embodiments, the tag is an optical tag, a chemical tag, or a biochemical tag. In some embodiments, the composition further comprises a magnetic element or a photonic element. In some embodiments, the complex is deconstructable upon application of a stimulus. In some embodiments, the complex further comprises a labile bond selected from a thioester bond, a disulfide bond, a carbamate bond, an amide bond, an ester bond, an acetal bond, an orthoester bond, a phosphoester bond, an anhydride bond, a hydrazone/Schiff base bond, an oxime bond, a glycosidic bond, an imine bond, and an acyl-hydrazone bond.

Provided herein are methods for processing a sequence-controlled polymer, comprising: (a) contacting the sequence-controlled polymer with a DMAEMA-based polymer or an AETAC-based polymer in an aqueous solution to form a first mixture; (b) removing the aqueous solution from the first mixture; and (c) contacting the first mixture with a hydrophobic agent to form a second mixture; wherein the second mixture comprises a hydrophobic polymer or polymer network that coats the sequence-controlled polymer. In some embodiments, the aqueous solution is water. In some embodiments, the method is performed between 25° C. and 100° C. In some embodiments, the sequence-controlled polymer is a nucleic acid molecule. In some embodiments, the nucleic acid molecule is DNA. In some embodiments, the nucleic acid molecule is RNA. In some embodiments, the sequence-controlled polymer is a peptide. In some embodiments, the sequence-controlled polymer is an enzyme. In some embodiments, the sequence-controlled polymer is a protein. In some embodiments, a barcode or a tag is attached to the coated sequence-controlled polymer. In some embodiments, the tag is an optical tag, a chemical tag, or a biochemical tag. In some embodiments, the hydrophobic agent is a hydrophobic monomer. In some embodiments, the hydrophobic agent is soluble in an organic solvent. In some embodiments, the hydrophobic agent is styrene. In some embodiments, the hydrophobic agent is DDA. In some embodiments, the DMAEMA-based polymer is a DMAEMA-co-OEGMA-co-styrene polymer. In some embodiments, the DMAEMA-co-OEGMA-co-styrene polymer has a ratio of DMAEMA: OEGMA: styrene of 1:1:1. In some embodiments, the AETAC-based polymer is an AETAC-co-OEGA-co-DDA polymer. In some embodiments, the AETAC-co-OEGA-co-DDA polymer has a ratio of AETAC: OEGA: DDA of 6:31:63. In some embodiments, the method further comprises (d) contacting the second mixture with a stimulus to form a third mixture; and (e) heating the third mixture, wherein heating the third mixture releases the sequence-controlled polymer. In some embodiments, the stimulus comprises light. In some embodiments, the stimulus comprises a deconstruction solution. In some embodiments, the deconstruction solution comprises a nucleophile, an organic base, or a polar organic solvent. In some embodiments, the nucleophile is cysteamine. In some embodiments, the organic base is 1,8-diazabicyclo[5.4.0]undec-7-ene (DBU) or propylamine. In some embodiments, the polar solvent is aprotic. In some embodiments, the polar solvent is a base. In some embodiments, the polar solvent is dimethylformamide (DMF) or propylamine. In some embodiments, the deconstruction solution comprises cysteamine, DBU, and DMF. In some embodiments, (d) and (e) release the sequence-controlled polymer in at most 5 hours. In some embodiments, (d) and (e) release the sequence-controlled polymer in at most 2 hours. In some embodiments, (d) and (e) release the sequence-controlled polymer in at most 1 hour. In some embodiments, the method further comprises (f) extracting the released sequence-controlled polymer from the third mixture. In some embodiments, the extraction comprises contacting the third mixture with an organic solvent. In some embodiments, the organic solvent is ethyl acetate. In some embodiments, the organic solvent is chloroform.

In some embodiments, in a), the sequence-controlled polymer and the DMAEMA-based polymer or the AETAC-based polymer form a complex in the first mixture, wherein the complex comprises: i) the sequence-controlled polymer; and ii) the DMAEMA-based polymer or the AETAC-based polymer. In some embodiments, the method further comprises, prior to or during e), deconstructing the complex. In some embodiments, in a), the complex comprises a labile bond selected from a thioester bond, a disulfide bond, a carbamate bond, an amide bond, an ester bond, an acetal bond, an orthoester bond, a phosphoester bond, an anhydride bond, a hydrazone/Schiff base bond, an oxime bond, a glycosidic bond, an imine bond, and an acyl-hydrazone bond; and deconstructing the complex prior to or during e) comprises breaking the labile bond.

Provided herein are methods for processing a sequence-controlled polymer, comprising: (a) creating an emulsion of: (i) the sequence-controlled polymer in an aqueous solution; and (ii) a DMAEMA-based polymer or an AETAC-based polymer in a hydrophobic agent; and (b) applying a first stimulus to initiate the formation of a hydrophobic polymer or polymer network that coats the sequence-controlled polymer within the emulsion. In some embodiments, the hydrophobic polymer or polymer network comprises hydrophobic polymer nanoparticles or microparticles. In some embodiments, the hydrophobic polymer nanoparticles have a dimension of at least 70 nm. In some embodiments, the hydrophobic polymer microparticles have a dimension of at most 1 mm. In some embodiments, the aqueous solution is water. In some embodiments, the method is performed between 25° C. and 100° C. In some embodiments, the sequence-controlled polymer is a nucleic acid molecule. In some embodiments, the nucleic acid molecule is DNA. In some embodiments, the nucleic acid molecule is RNA. In some embodiments, the sequence-controlled polymer is a peptide. In some embodiments, the sequence-controlled polymer is an enzyme. In some embodiments, the sequence-controlled polymer is a protein. In some embodiments, a barcode or a tag is attached to the coated sequence-controlled polymer. In some embodiments, the tag is an optical tag, a chemical tag, or a biochemical tag. In some embodiments, the hydrophobic agent is a hydrophobic monomer. In some embodiments, the hydrophobic agent is soluble in an organic solvent. In some embodiments, the hydrophobic agent is styrene. In some embodiments, the hydrophobic agent is DDA. In some embodiments, the DMAEMA-based polymer is a DMAEMA-co-OEGMA-co-styrene polymer. In some embodiments, the DMAEMA-co-OEGMA-co-styrene polymer has a ratio of DMAEMA: OEGMA: styrene of 1:1:1. In some embodiments, the AETAC-based polymer is an AETAC-co-OEGA-co-DDA polymer. In some embodiments, the AETAC-co-OEGA-co-DDA polymer has a ratio of AETAC:OEGA:DDA of 6:31:63. In some embodiments, the first stimulus is light. In some embodiments, the first stimulus is heat. In some embodiments, the method further comprises (c) applying a second stimulus to the emulsion, thereby separating the hydrophobic agent from the emulsion and forming a hydrophobic polymer or polymer network that coats the sequence-controlled polymer. In some embodiments, the second stimulus is light. In some embodiments, the second stimulus is heat. In some embodiments, the method further comprises (d) extracting the released sequence-controlled polymer from the hydrophobic polymer or polymer network. In some embodiments, the extraction comprises contacting the third mixture with an organic solvent. In some embodiments, the organic solvent is ethyl acetate. In some embodiments, the organic solvent is chloroform.

In some embodiments, in a), the sequence-controlled polymer and the DMAEMA-based polymer or the AETAC-based polymer form a complex in the emulsion, wherein the complex comprises: i) the sequence-controlled polymer; and ii) the DMAEMA-based polymer or the AETAC-based polymer. In some embodiments, the method further comprises, after a), deconstructing the complex. In some embodiments, in a), the complex comprises a labile bond selected from a thioester bond, a disulfide bond, a carbamate bond, an amide bond, an ester bond, an acetal bond, an orthoester bond, a phosphoester bond, an anhydride bond, a hydrazone/Schiff base bond, an oxime bond, a glycosidic bond, an imine bond, and an acyl-hydrazone bond; and deconstructing the complex after a) comprises breaking the labile bond.

Provided herein are kits for storage and retrieval of a sequence-controlled polymer, comprising: (a) a flowcell comprising a support configured to bind the sequence-controlled polymer to the support; (b) an amphiphilic polymer; (c) a hydrophilic agent; and (d) a hydrophobic agent. In some embodiments, the amphiphilic polymer is an amine-containing copolymer. In some embodiments, the amine-containing copolymer is DMAEMA. In some embodiments, the amphiphilic polymer is a cation-containing copolymer. In some embodiments, the cation-containing copolymer is AETAC. In some embodiments, the hydrophilic agent is OEGMA. In some embodiments, the hydrophilic agent is OEGA. In some embodiments, the hydrophobic agent is a hydrophobic monomer. In some embodiments, the hydrophobic agent is soluble in an organic solvent. In some embodiments, the hydrophobic agent is styrene. In some embodiments, the hydrophobic agent is DDA. In some embodiments, the kit further comprises instructions for use. In some embodiments, the kit further comprises an aqueous solution. In some embodiments, the aqueous solution comprises a nucleophile, an organic base, or a polar organic solvent. In some embodiments, the nucleophile is cysteamine. In some embodiments, the organic base is 1,8-diazabicyclo[5.4.0]undec-7-ene (DBU) or propylamine. In some embodiments, the polar solvent is aprotic. In some embodiments, the polar solvent is a base. In some embodiments, the polar solvent is dimethylformamide (DMF) or propylamine. In some embodiments, the aqueous solution comprises cysteamine, DBU, and DMF.

Additional aspects and advantages of the present disclosure will become readily apparent to those skilled in this art from the following detailed description, wherein only illustrative embodiments of the present disclosure are shown and described. As will be realized, the present disclosure is capable of other and different embodiments, and its several details are capable of modifications in various obvious respects, all without departing from the disclosure. Accordingly, the drawings and description are to be regarded as illustrative in nature, and not as restrictive.

### INCORPORATION BY REFERENCE

All publications, patents, and patent applications mentioned in this specification are herein incorporated by reference to the same extent as if each individual publication, patent, or patent application was specifically and individually indicated to be incorporated by reference. To the extent publications and patents or patent applications incorporated by reference contradict the disclosure contained in the specification, the specification is intended to supersede and/or take precedence over any such contradictory material.

### BRIEF DESCRIPTION OF THE DRAWINGS

The novel features of the invention are set forth with particularity in the appended claims. A better understanding of the features and advantages of the present invention will be obtained by reference to the following detailed description that sets forth illustrative embodiments, in which the principles of the invention are utilized, and the accompanying drawings (also “Figure” and “FIG.” herein), of which:

FIG. 1 illustrates an exemplary schematic representation of the encapsulation process and deconstruction process for sequence-controlled polymers within deconstructable thermoset matrices.

FIG. 2A presents optical micrographs of polymerized deconstructable polystyrene containing DNA polyplexes. The physical characteristics of these DNA-embedded deconstructable thermosets are found to be analogous to those of deconstructable thermosets devoid of DNA content. FIG. 2B illustrates the disassembly of polystyrene, enabling the efficient release of the encapsulated DNA into the surrounding solution for subsequent processing.

FIG. 3A presents a line chart that illustrates the absorbance spectra of DNA samples procured from deconstructable thermosets, both before and after the desalting process employing ethanol precipitation. FIG. 3B showcases a line chart that portrays the analytical examination of the sequencing fragments produced from the DNA sample depicted in FIG. 3A.

FIG. 4 illustrates a schematic representation of the polyplex-mediated transfer of DNA to organic solvents through emulsion.

FIG. 5A presents optical micrographs that track the HEX-labelled DNA in water/organic solvent biphasic systems. FIG. 5B presents optical micrographs of dried polyplexes that are successfully resuspended in tert-butyl acrylate, hexanes, and ethyl acetate. FIG. 5C presents optical micrographs that tracks the HEX-labelled DNA in water/organic solvent biphasic systems. Addition of SDS successfully re-extracts DNA into the aqueous layer (turns pink).

FIG. 6A presents optical micrographs that track the HEX-labelled DNA in a water/styrene biphasic system. FIGS. 6B-6C present DLS characterization of 40 mer ssDNA, polymer, and polyplex, showing the formation of polyplexes with Rₕ˜80 nm in emulsion transfer. FIG. 6B shows the normalized intensity percent plot. FIG. 6C shows the normalized mass percent plot.

FIG. 7 presents MALDI-TOF-MS of the HEX-labelled 40 mer before encapsulation (top) and after encapsulation and release (bottom).

FIG. 8A presents UV-Vis absorbance spectra of 40 bp dsDNA dissolved in water, and of sample solutions at various time points during transfer to styrene through emulsion. FIGS. 8B-8C present DLS characterization of 40 mer ssDNA, 40 bp dsDNA, polymer, and polyplex, showing the formation of polyplexes with Rₕ˜75 nm in emulsion transfer. FIG. 8B shows the normalized intensity percent plot. FIG. 8C shows the normalized mass percent plot. FIG. 8D presents optical micrographs showing the curing of the deconstructable thermoset.

FIG. 9A presents DLS characterization of polymer and polyplex, showing that polyplexes comprised of genomic DNA has a larger Rₕ˜230 nm. FIG. 9B presents a table comparing the effectiveness of different N/P ratios in mediating the transfer and encapsulation of high-MW genomic DNA. FIG. 9C is a fragment analysis trace of the sequencing library generated from the extracted genomic DNA. FIG. 9D shows low-pass sequencing data and sequencing coverage of human DNA.

FIG. 10 shows an exemplary thermoset formation using poly(styrene-co-divinylbenzene) and thermoset deconstruction using cysteamine HCl and DBU.

FIG. 11A shows normalized fluorescence of DNA, polyplex in water, and polyplex in styrene. FIG. 11B shows mass fraction percent of DNA, polymer, polyplex in water, and polyplex in styrene. FIG. 11C shows normalized absorbance of DNA, polyplex in water, and polyplex in styrene.

FIGS. 12A-12E show examples of polymer combinations at different ratios for the formation of the thermoset. Tested polymer combinations included p(DMAEMA-co-OEGMA) at a ratio of 1:1 (FIG. 12A), p(DMAEMA-co-styrene) at a ratio of 1:1 (FIG. 12B); p(DMAEMA-co-styrene) at a ratio of 1:4 (FIG. 12C); p(DMAEMA) (FIG. 12D); and p(DMAEMA-co-OEGMA-co-styrene) at a ratio of 1:1:1 (FIG. 12E).

FIG. 13A shows normalized fluorescence data during the synthesis of polyplex polymers. FIG. 13B shows a summary of the data of FIG. 13A where f is the ratio in feed and F is the ratio in polymer.

FIG. 14 illustrates a thermoset being barcoded with a fluorescent probe.

FIG. 15A depicts a schematic of a microfluidic device used to synthesize thermosets. FIG. 15B shows an exemplary microfluidic device. FIG. 15C is an enlarged portion of FIG. 15B where the three liquid channels merge.

FIG. 16A shows the synthesis of p(AETAC-co-OEGA-co-DDA). FIG. 16B shows a summary of characterization data (NMR and SEC-MALS) of p(AETAC-co-OEGA-co-DDA). ^{a}Ratio in polymer (F) determined via ¹H NMR (conversion >99.5%. ^{b}Determined via SEC-MALS, dn/dc values obtained from full mass recovery method, eluent: 0.025 M LiBr in DMF. ^{c}All polymers are soluble in water and styrene at >10 mg/mL.

FIG. 17 shows example schemes of furnishing polycations with a cleavable bond to render a polyplex degradable during polymer network deconstruction.

FIG. 18A shows an example scheme to evaluate polycation performance in reversibly shuttling DNA between water and styrene. FIG. 18B shows an example scheme of polycation synthesis. FIG. 18C shows forward extraction and back extraction data for 200 bp DNA. FIG. 18D shows forward extraction and back extraction data for human genomic DNA (>50,000 bp).

FIG. 19A shows photos depicting the effect of the polyplex on the curing of the styrene-divinylbenzene network (with or without DOT). FIG. 19B shows decomposition temperature at inflection point data. FIG. 19C shows glass transition temperature data.

FIG. 20A shows an example scheme depicting synthesis and deconstruction of a styrene-divinylbenzene network installed with thioester cleavable bonds. FIG. 20B shows photos depicting the effect of inclusion of DOT and the polyplex on deconstruction of the styrene-divinylbenzene network. FIG. 20C shows fluorescence spectra of HEX-labeled DNA, the polyplex, and deconstruction products. FIG. 20D shows MALDI-TOF mass spectra of the HEX-labeled DNA as purchased and the HEX-labeled DNA following encapsulation and retrieval from the network.

FIG. 21A shows various polymerization conditions tested and the corresponding oligonucleotide stability results evaluated by MALDI-TOF MS. FIG. 21B shows example MALDI-TOF mass spectra of an 8-base oligomer after testing in various polymerization conditions.

### DETAILED DESCRIPTION

While various embodiments of the invention have been shown and described herein, it will be obvious to those skilled in the art that such embodiments are provided by way of example only. Numerous variations, changes, and substitutions may occur to those skilled in the art without departing from the invention. It should be understood that various alternatives to the embodiments of the invention described herein may be employed.

Large-scale storage of nucleic acid samples is critical in basic, translational, and clinical research, synthetic biology foundries, and biodiversity conservation efforts. Nucleic acid storage uses robust procedures to maintain sample quality, integrity, and function. The storage temperature requirement for nucleic acids can be between 4° C. to −196° C., where degradation is negligible. However, maintaining such a low temperature for extended periods requires significant energy. Also, large-scale cryogenic storage of nucleic acid materials requires extensive robotics for access, stringent cold-chain management logistics, and redundant copies of samples stored in mirror storage facilities to mitigate the risk of sample loss. Cold storage of nucleic acids in remote or low-resource areas will involve costly measures and complex cold-chain logistics to maintain the integrity and quality of an isolated sample during transport.

A transition towards room-temperature storage from cryogenic storage can reduce energy usage by 40 million kilowatt-hours, which translates to eliminating 18,000 metric tons of annual carbon dioxide emissions and cost savings of $16 million over ten years, and 70% reduction of space requirements over cryogenic storage. The cost and workflow complexity associated with sample processing can also be reduced. Room-temperature storage of nucleic acid samples can be achieved through the addition of stabilizing agents, or the use of vacuum canisters. While these room-temperature storage solutions can guarantee nucleic acid stability of one year or more, space to store samples and support infrastructure, such as extensive robotic platforms for access and humidity controls, will still be critical cost considerations.

While silica particles, alginates, and synthetic polymers have been used for storing biomolecules at room temperature, long encapsulation times and use of extremely corrosive chemicals, e.g., hydrofluoric acid, have limited the utility of silica for long-term storage of nucleic acids.

There is a need for scalable storage of biomolecules that can use little to no energy for maintaining the integrity of samples over 10 years or more. There is also a need to reduce the footprint used to store biomolecule samples and be able to retrieve thousands to millions of samples rapidly. Therefore, the object of the compositions, methods, and systems described here is to store and retrieve biomolecules collected from any origin and to encapsulate biomolecules of various lengths and sizes using different chemical and biochemical preparations and different fluidic approaches.

Presented herein is alternative encapsulation chemistry using hydrophobic polymers or polymer networks to realize a room-temperature storage and retrieval approach for biomolecules, e.g., DNA, RNA, or proteins, using hydrophobic biomolecules. The disclosed technology can be applied to storage and retrieval of synthetic and naturally derived biomolecules.

Purified nucleic acids from any origin may be encapsulated in synthetic packets composed of organic or inorganic hydrophobic polymeric networks. Encapsulation may be performed using automated liquid handling, which mixes the biomolecules of interest with encapsulation reagents, or millifluidic and microfluidic approaches, which traps biomolecules and encapsulates reagents in millimeter to nanometer-sized emulsion reaction containers. The encapsulated biomolecules may be labeled with combinations of orthogonal molecular barcodes identified from a pool of 240,000, which may label and identify the contents of the sample. The molecular barcodes may comprise non-phosphate backbones to improve the stability of strands against nucleases. The process of barcoding can be similarly performed using millifluidic or microfluidic approaches. Upon encapsulation and barcoding, all samples can be collected and pooled into a single vessel. Samples can be selected from the pool using complementary probes which may contain optical, chemical, or biochemical tags that can be used as markers for downstream optical or mechanical sorting using millifluidic or microfluidic strategies. Chemical and biochemical reactions can be performed on the barcodes to improve the sorting speed, sorting precision, and limit-of-detection of a specific sorting approach.

Nucleic acids can be stored at −20 to −80° C. to maintain their long-term integrity. Such methods are energy-intensive, difficult-to-scale, and inaccessible to resource-poor areas. With the expanding use of DNA in genomics, synthetic biology, conservation, and nanotechnology, there is a need for developing more sustainable and cost-effective methods for storing large amounts of DNA at ambient conditions. Herein, a strategy was developed for storing DNA inside a deconstructable thermoset that can serve to protect the encapsulated nucleic acids against biological, chemical, or thermal degradation. Implementation of this strategy can use a cationic, amphiphilic polymer that complexes with DNA, resulting in the formation of nano-polyplexes that can be suspended in neat styrene. Subsequent radical polymerization of styrene with a cleavable comonomer additive, e.g., dibenzo[c,e]oxepine-5(7H)-thione derivatives, can generate a deconstructable polystyrene (PS) network that can preserve polystyrene's desirable material properties (e.g., no decrease in thermal decomposition and glass transition temperatures compared to native, DNA-free PS network). Notably, the encapsulation procedure takes less than about 2 days, significantly faster than silica encapsulation (4 days); network deconstruction and DNA retrieval can be completed in about 12 hours with cysteamine and DBU, which are less hazardous and easier to handle than silica etchant, HF.

The polyplex (e.g., polycation-DNA complex) can optionally be deconstructed. In some cases, the polyplex is deconstructable upon application of a stimulus. For example, a polycation can be furnished with a degradable labile bond or a reversible covalent bond. A labile bond can be a thioester bond, a disulfide bond, a carbamate bond, an amide bond, an ester bond, an acetal bond, an orthoester bond, a phosphoester bond, an anhydride bond, a hydrazone/Schiff base bond, an oxime bond, a glycosidic bond, an imine bond, and an acyl-hydrazone bond. The labile bond can be broken in the presence of an enzyme, a nucleophile, a small molecule, a thermal stimulus, or a light stimulus. In some cases, for polycations used in polyplexes, the cationic moiety is located at or adjacent to the cleavable site to facilitate breakdown of the polycations to monocationic fragments. In some cases, the methods described herein result in complete breakdown of the polycations in the polyplexes to monocationic fragments. The polyplex can be degraded during polymer network deconstruction. In some cases, the polyplex is degraded by the same stimulus used to deconstruct the polymer network. Alternatively, two or more different stimuli can be applied to degrade the polyplex and to deconstruct the polymer network. The two or more different stimuli can be applied simultaneously or sequentially.

Importantly, the retrieved DNA are chemically intact (as verified by mass spectrometry). This strategy can be directly applied in DNA-based archival data storage.

The disclosed storage and retrieval system can isolate the biomolecule of interest from the environment to protect the integrity of the biomolecule over ten years or longer and reduces or eliminates the need for low-temperature storage conditions. Barcoding micron-to-nanoscale capsules enables the pooling of all samples in a single vessel rather than millions of individual tubes, thus reducing the footprint of biomolecular storage to size dimensions that can sit on top of a desktop.

### Definitions

The terms “nucleic acid,” “nucleic acid molecule,” a “nucleic acid fragment,” “oligonucleotide” and “polynucleotide” are used interchangeably and are intended to include, but not limited to, a polymeric form of nucleotides that may have various lengths, either deoxyribonucleotides (DNA) or ribonucleotides (RNA), or analogs or modified nucleotides thereof, including, but not limited to locked nucleic acids (LNA) and peptide nucleic acids (PNA). An oligonucleotide is typically composed of a specific sequence of four nucleotide bases: adenine (A); cytosine (C); guanine (G); and thymine (T) (uracil (U) for thymine (T) when the polynucleotide is RNA). Thus, the term “oligonucleotide sequence” is the alphabetical representation of a polynucleotide molecule; alternatively, the term may be applied to the polynucleotide molecule itself. This alphabetical representation can be input into databases in a computer having a central processing unit and used for bioinformatics applications such as functional genomics and homology searching. Oligonucleotides may optionally include one or more non-standard nucleotide(s), nucleotide analog(s) and/or modified nucleotides.

The terms “encapsulating”, “enveloping”, “coating”, “covering”, and “shelling” are used interchangeably to refer to the process by which biomolecules are completely or partially enclosed by an encapsulating agent. The term “encapsulating agent” refers to a molecular entity, such as a polymer or other matrix.

Whenever the term “at least,” “greater than,” or “greater than or equal to” precedes the first numerical value in a series of two or more numerical values, the term “at least,” “greater than” or “greater than or equal to” applies to each of the numerical values in that series of numerical values. For example, greater than or equal to 1, 2, or 3 is equivalent to greater than or equal to 1, greater than or equal to 2, or greater than or equal to 3.

Whenever the term “no more than,” “less than,” or “less than or equal to” precedes the first numerical value in a series of two or more numerical values, the term “no more than,” “less than,” or “less than or equal to” applies to each of the numerical values in that series of numerical values. For example, less than or equal to 3, 2, or 1 is equivalent to less than or equal to 3, less than or equal to 2, or less than or equal to 1.

### Biomolecule Storage

A biomolecule can be stored in a hydrophobic polymer or thermoset composition through the creation of an emulsion that coats the biomolecule and sets it into a hydrophobic polymer or polymer network.

A thermoset composition can comprise an amphiphilic polymer. A thermoset composition can comprise a hydrophilic agent. A thermoset composition can comprise a hydrophobic agent. A thermoset composition can comprise an amphiphilic polymer and a hydrophilic agent. A thermoset composition can comprise an amphiphilic polymer and a hydrophobic agent. A thermoset composition can comprise a hydrophilic agent and a hydrophobic agent. A thermoset composition can comprise an amphiphilic polymer, a hydrophilic agent, and a hydrophobic agent.

A thermoset composition can comprise one amphiphilic polymer. Alternatively, a thermoset composition can comprise at least 2, 3, 4, 5, 6, 7, 8, 9, 10, or more different amphiphilic polymers. A thermoset composition can comprise one hydrophilic agent. Alternatively, a thermoset composition can comprise at least 2, 3, 4, 5, 6, 7, 8, 9, 10, or more different hydrophilic agents. A thermoset composition can comprise one hydrophobic agent. Alternatively, a thermoset composition can comprise at least 2, 3, 4, 5, 6, 7, 8, 9, 10, or more different hydrophobic agents.

A thermoset composition can comprise its components (e.g., one or more amphiphilic polymers, one or more hydrophilic agents, and/or one or more hydrophobic agents) in a specified ratio. The ratio of any two components in a thermoset composition can be at least about 1:1, at least about 2:1, at least about 3:1, at least about 4:1, at least about 5:1, at least about 6:1, at least about 7:1, at least about 8:1, at least about 9:1, at least about 10:1, at least about 11:1, at least about 12:1, at least about 13:1, at least about 14:1, at least about 15:1, at least about 16:1, at least about 17:1, at least about 18:1, at least about 19:1, at least about 20:1 or more.

The ratio of an amphiphilic polymer to a hydrophilic agent in a thermoset composition can be at least about 1:1, at least about 2:1, at least about 3:1, at least about 4:1, at least about 5:1, at least about 6:1, at least about 7:1, at least about 8:1, at least about 9:1, at least about 10:1, at least about 11:1, at least about 12:1, at least about 13:1, at least about 14:1, at least about 15:1, at least about 16:1, at least about 17:1, at least about 18:1, at least about 19:1, at least about 20:1 or more. For example, the ratio of an amphiphilic polymer to a hydrophilic agent can be 1:1. Alternatively, the ratio of an amphiphilic polymer to a hydrophilic agent can be 6:94.

The ratio of an amphiphilic polymer to a hydrophobic agent in a thermoset composition can be at least about 1:1, at least about 2:1, at least about 3:1, at least about 4:1, at least about 5:1, at least about 6:1, at least about 7:1, at least about 8:1, at least about 9:1, at least about 10:1, at least about 11:1, at least about 12:1, at least about 13:1, at least about 14:1, at least about 15:1, at least about 16:1, at least about 17:1, at least about 18:1, at least about 19:1, at least about 20:1 or more. For example, the ratio of an amphiphilic polymer to a hydrophobic agent can be 1:1. Alternatively, the ratio of an amphiphilic polymer to a hydrophobic agent can be 6:94.

The ratio of a hydrophilic agent to a hydrophobic agent in a thermoset composition can be at least about 1:1, at least about 2:1, at least about 3:1, at least about 4:1, at least about 5:1, at least about 6:1, at least about 7:1, at least about 8:1, at least about 9:1, at least about 10:1, at least about 11:1, at least about 12:1, at least about 13:1, at least about 14:1, at least about 15:1, at least about 16:1, at least about 17:1, at least about 18:1, at least about 19:1, at least about 20:1 or more. For example, the ratio of a hydrophilic agent to a hydrophobic agent can be 1:1. Alternatively, the ratio of a hydrophilic agent to a hydrophobic agent can be 6:94.

The ratio of an amphiphilic polymer to a hydrophilic agent to a hydrophobic agent can be at least about 1:1:1. Alternatively, the ratio of an amphiphilic polymer to a hydrophilic agent to a hydrophobic agent can be at least about 6:74:20. Alternatively, the ratio of an amphiphilic polymer to a hydrophilic agent to a hydrophobic agent can be at least about 6:62:31. Alternatively, the ratio of an amphiphilic polymer to a hydrophilic agent to a hydrophobic agent can be at least about 6:54:40. Alternatively, the ratio of an amphiphilic polymer to a hydrophilic agent to a hydrophobic agent can be at least about 6:31:63.

An amphiphilic polymer is a polymer that is comprised of a hydrophilic and a hydrophobic component. Amphiphilic polymers can be polymers which can self-assemble (e.g., into a micelle, a vesicle, a hydrogel, etc.) when dispersed in water. Amphiphilic polymers can have various architectures including but not limited to block architecture, grafted architecture, cyclic architecture, star-shaped architecture, dendritic architecture, comb-like architecture, or hyperbranched architecture. Amphiphilic polymers can contain amines (e.g., 2-(dimethylamino)ethyl methacrylate (DMAEMA), allylamine, iminoethylene, lysine, and their derivatives). Alternatively or in addition to, amphiphilic polymers can contain cations (e.g., [2-(acryloyloxy)ethyl]trimethylammonium chloride (AETAC), diallyldimethylmine hydrochloride, arginine hydrochloride, and their derivatives).

A hydrophilic agent, also known as a hydrophilic substance, is a substance that is capable of interacting with water through hydrogen bonding. Non-limiting examples of hydrophilic agents include oligo(ethylene glycol) methyl ether methacrylate (OEGMA), PEG methacrylate (PEGMA), and oligo(ethylene glycol) methyl ether acrylate (OEGA).

A hydrophobic agent, also known as a hydrophobic substance, is a substance that is not capable of interacting with water through hydrogen bonding. Hydrophobic agents can be soluble in organic solvents. Non-limiting examples of hydrophobic agents include styrene, methyl methacrylate (MMA), and dodecyl acrylate (DDA).

A thermoset composition can be a composition comprising DMAEMA, OEGMA, and styrene. For example, a thermoset composition can comprise DMAEMA-co-OEGMA-co-styrene polymer. Alternatively, a thermoset composition can be a composition comprising AETAC, OEGA, and DDA. For example, a thermoset composition can comprise AETAC-co-OEGA-co-DDA polymer. Alternatively, a thermoset composition can be a composition comprising AETAC, PEGMA, and DDA. For example, a thermoset composition can comprise AETAC-co-PEGMA-co-DDA polymer.

A thermoset composition comprising an amphiphilic polymer, a hydrophilic agent, and/or a hydrophobic agent can comprise a hydrophobic polymer network. A hydrophobic polymer network can be porous. Alternatively, a hydrophobic polymer network can be non-porous.

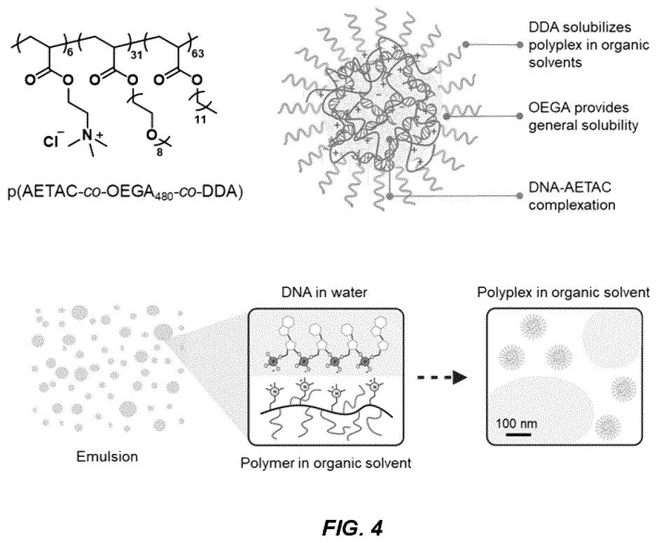

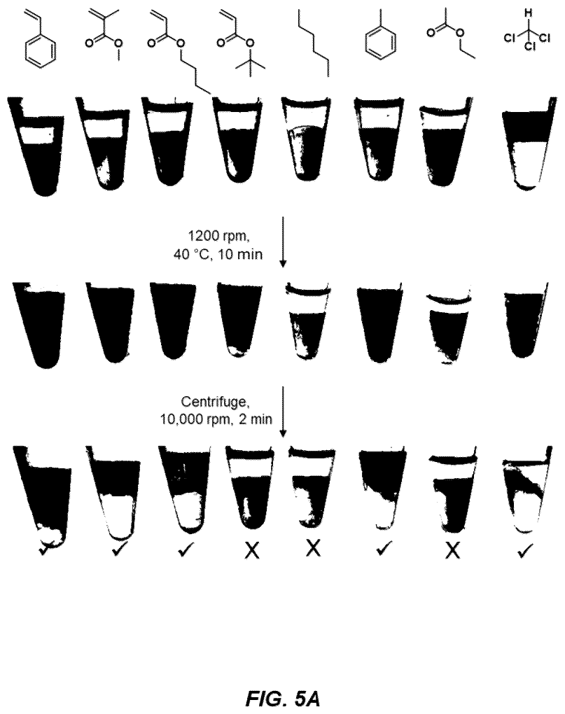

A hydrophobic polymer or polymer network can comprise a non-aqueous solution such as an organic solvent. Non-limiting examples of organic solvents can be styrene, methyl methacrylate, n-butyl acrylate, tert-butyl acrylate, hexanes, toluene, ethyl acetate, and chloroform. A hydrophobic polymer or polymer network can be formed through the creation of an emulsion. For example, FIG. 4 shows an organic-soluble polycation neutralize the negative charge of DNA and mediate the transfer of the latter to organic solvent through ion-pairing and polycation-polyanion complexation, resulting in the formation of nano- or micron-scale polyplex nanoparticles or microparticles.

A thermoset composition can comprise additional substances including, but not limited to proteins, lipids, saccharides, polysaccharides, nucleic acids, synthetic polymers, hydrogel polymers, silica, magnetic elements, paramagnetic materials, photonic elements, metals, or derivatives thereof. Additional substances in a thermoset composition can provide additional stability or other beneficial properties.

A hydrophobic polymer or polymer network can comprise hydrophobic polymer nanoparticles or microparticles. Hydrophobic polymer nanoparticles or microparticles can have a greatest dimension of at least about 30 nanometers (nm), at least about 40 nm, at least about 50 nm, at least about 60 nm, at least about 70 nm, at least about 80 nm, at least about 90 nm, at least about 100 nm, at least about 200 nm, at least about 300 nm, at least about 400 nm, at least about 500 nm, at least about 600 nm, at least about 700 nm, at least about 800 nm, at least about 900 nm, at least about 1 micrometer (μm), at least about 10 μm, at least about 100 μm, at least about 200 μm, at least about 300 μm, at least about 400 μm, at least about 500 μm, at least about 600 μm, at least about 700 μm, at least about 800 μm, at least about 900 μm, at least about 1 millimeter (mm), at least about 2 mm, at least about 3 mm, at least about 4 mm, at least about 5 mm, at least about 6 mm, at least about 7 mm, at least about 8 mm, at least about 9 mm, or at least about 10 mm. Hydrophobic polymer nanoparticles or microparticles can have a greatest dimension of at most about 10 mm, at most about 9 mm, at most about 8 mm, at most about 7 mm, at most about 6 mm, at most about 5 mm, at most about 4 mm, at most about 3 mm, at most about 2 mm, at most about 1 mm, at most about 900 μm, at most about 800 μm, at most about 700 μm, at most about 600 μm, at most about 500 μm, at most about 400 μm, at most about 300 μm, at most about 200 μm, at most about 100 μm, at most about 10 μm, at most about 1 μm, at most about 900 nm, at most about 800 nm, at most about 700 nm, at most about 600 nm, at most about 500 nm, at most about 400 nm, at most about 300 nm, at most about 200 nm, at most about 100 nm, at most about 90 nm, at most about 80 nm, at most about 70 nm, at most about 60 nm, at most about 50 nm, at most about 40 nm, or at most about 30 nm.

A hydrophobic polymer or polymer network can form at a temperature of at least about 5° C., at least about 10° C., at least about 15° C., at least about 20° C., at least about 25° C., at least about 30° C., at least about 35° C., at least about 40° C., at least about 45° C., at least about 50° C., at least about 55° C., at least about 60° C., at least about 65° C., at least about 70° C., at least about 75°°C., at least about 80° C., at least about 85° C., at least about 90° C., at least about 95° C., at least about 100° C., at least about 105° C., at least about 110° C., at least about 115° C., at least about 120° C., at least about 125° C., at least about 130° C., at least about 135° C., at least about 140° C., at least about 145° C., or at least about 150° C. A hydrophobic polymer network can form at a temperature of at most about 150° C., at most about 145° C., at most about 140° C., at most about 135° C., at most about 130° C., at most about 125° C., at most about 120° C., at most about 115° C., at most about 110° C., at most about 105° C., at most about 100° C., at most about 95° C., at most about 90° C., at most about 85° C., at most about 80° C,, at most about 75° C., at most about 70° C., at most about 65° C., at most about 60° C., at most about 55° C., at most about 50° C., at most about 45° C., at most about 40° C., at most about 35° C., at most about 30° C., at most about 25° C., at most about 20° C., at most about 15° C., at most about 10° C., or at most about 5° C.

A hydrophobic polymer or polymer network can form under the irradiation of light. The light source can be of UV light or visible light.

A hydrophobic polymer or polymer network can be stable at a temperature of at least about 5° C., at least about 10° C., at least about 15° C., at least about 20° C., at least about 25° C., at least about 30° C., at least about 35° C., at least about 40° C., at least about 45° C., at least about 50° C., at least about 55° C., at least about 60° C., at least about 65° C., at least about 70° C., at least about 75° C., at least about 80° C., at least about 85° C., at least about 90° C., at least about 95° C., at least about 100° C., at least about 105° C., at least about 110° C., at least about 115° C., at least about 120° C., at least about 125° C., at least about 130° C., at least about 135° C., at least about 140° C., at least about 145° C., or at least about 150° C. A hydrophobic polymer or polymer network can be stable at a temperature of at most about 150° C., at most about 145° C., at most about 140° C., at most about 135° C., at most about 130° C., at most about 125° C., at most about 120° C., at most about 115° C., at most about 110° C., at most about 105° C., at most about 100° C., at most about 95° C., at most about 90° C., at most about 85° C., at most about 80° C., at most about 75° C., at most about 70° C., at most about 65° C., at most about 60° C., at most about 55° C., at most about 50° C., at most about 45° C., at most about 40° C., at most about 35° C., at most about 30° C., at most about 25° C., at most about 20° C., at most about 15° C., at most about 10° C., or at most about 5° C.

A hydrophobic polymer or polymer network can be stable for at least about 1 week, at least about 2 weeks, at least about 3 weeks, at least about 1 month, at least about 2 months, at least about 3 months, at least about 4 months, at least about 5 months, at least about 6 months, at least about 7 months, at least about 8 months, at least about 9 months, at least about 10 months, at least about 11 months, at least about 1 year, at least about 2 years, at least about 3 years, at least about 4 years, at least about 5 years, at least about 6 years, at least about 7 years, at least about 8 years, at least about 9 years, at least about 10 years, at least about 15 years, at least about 20 years, at least about 25 years, at least about 30 years, at least about 40 years, at least about 50 years, at least about 60 years, at least about 70 years, at least about 80 years, at least about 90 years, at least about 100 years, at least about 150 years, at least about 200 years, or more.

A biomolecule can be a sequenced-controlled polymer. A sequenced-controlled polymer is a macromolecule in which the sequence of the monomers is controlled to some degree. A sequence-controlled polymer is a macromolecule composed of two or more distinct monomer units sequentially arranged in a specific, non-random manner, as a polymer “chain.” The arrangement of two or more distinct monomer units constitutes a precise molecular “signature”, or “code” within the polymer chain. Sequence-controlled polymers can be biological polymers (e.g., biopolymers), or synthetic polymers. Exemplary sequence-controlled biopolymers include nucleic acids, polypeptides or proteins, linear or branched carbohydrate chains, or other sequence-controlled polymers that encode a format of information. Alternatively, a biomolecule can be a macromolecule such as but not limited to a carbohydrate or a lipid.

A sequenced-controlled polymer can be made of monomers. A sequence-controlled polymer can be single-stranded or double-stranded. A sequence-controlled polymer can be at least about 100, at least about 500, at least about 1,000, at least about 5,000, at least about 10,000, at least about 50,000, at least about 100,000, at least about 500,000, at least about 1,000,000, or more monomers in length.

A barcode or tag can be attached to a biomolecule. Non-limiting examples of tags can be optical tags, chemical tags, or biochemical tags. A barcode or tag can be attached to a biomolecule prior to coating with a thermoset composition, or a precursor thereof. Alternatively, or in addition to, a barcode or tag can be attached to a biomolecule after coating with a thermoset composition, or a precursor thereof. A barcode or tag can be attached covalently or non-covalently.

### Biomolecule Release from Storage

A biomolecule that has been stored in a thermoset composition can be released from the thermoset composition. A biomolecule that has been stored in a thermoset composition can be released spontaneously from the thermoset composition. Alternatively, a biomolecule that has been stored in a thermoset composition can be released from the thermoset composition upon encountering a stimulus. A stimulus can comprise light, heat, and/or a deconstruction solution.

A deconstruction solution can comprise a nucleophile. A deconstruction solution can comprise an organic base. A deconstruction solution can comprise a polar organic solvent. A deconstruction solution can comprise a nucleophile and an organic base. A deconstruction solution can comprise a nucleophile and a polar organic solvent. A deconstruction solution can comprise an organic base and a polar organic solvent. A deconstruction solution can comprise a nucleophile, an organic base, and a polar organic solvent.

A deconstruction solution can comprise one nucleophile. Alternatively, a deconstruction solution can comprise at least 2, at least 3, at least 4, at least 5, at least 6, at least 7, at least 8, at least 9, at least 10, or more different nucleophiles. A deconstruction solution can comprise one organic base. Alternatively, a deconstruction solution can comprise at least 2, at least 3, at least 4, at least 5, at least 6, at least 7, at least 8, at least 9, at least 10, or more different organic bases. A deconstruction solution can comprise one polar organic solvent. Alternatively, a deconstruction solution can comprise at least 2, at least 3, at least 4, at least 5, at least 6, at least 7, at least 8, at least 9, at least 10, or more different polar organic solvents.

A nucleophile is a molecule in a chemical reaction that comprises an electron pair available for bonding. An example of a nucleophile is cysteamine (e.g., cysteamine hydrochloride) and N-propylamine.

An organic base is a compound which acts as a base. An organic base can be a proton acceptor. Non-limiting examples of organic bases include 1,8-diazabicyclo[5.4.0]undec-7-ene (DBU) and N,N-diisopropylamine.

A polar organic solvent is a solvent that has both a positive and negative charge at different places in its structure and which can dissolve other polar substances. A polar organic solvent can be aprotic. A polar organic solvent can be a base. Non-limiting examples of polar organic solvents include acetone, acetonitrile, dimethylformamide (DMF), dimethylsufoxide (DMSO), chloroform, methyl ethyl ketone, 1-methyl-2-pyrrolidinone (NMP), dimethylacetamide, tetrahydrofuran (THF), dioxane, 1,2-dichloroethane, and dichloromethane (DCM).

A deconstruction solution can comprise cysteamine, DBU, and DMF.

A hydrophobic polymer network can be deconstructable upon application of a stimulus in at least about 5 minutes, at least about 10 minutes, at least about 20 minutes, at least about 30 minutes, at least about 40 minutes, at least about 50 minutes, at least about 1 hour, at least about 2 hours, at least about 3 hours, at least about 4 hours, at least about 5 hours, at least about 6 hours, at least about 7 hours, at least about 8 hours, at least about 9 hours, or at least about 10 hours. A hydrophobic polymer network can be deconstructable upon application of a stimulus in at most about 10 hours, at most about 9 hours, at most about 8 hours, at most about 7 hours, at most about 6 hours, at most about 5 hours, at most about 4 hours, at most about 3 hours, at most about 2 hours, at most about 1 hour, at most about 50 minutes, at most about 40 minutes, at most about 30 minutes, at most about 20 minutes, at most about 10 minutes, or at most about 5 minutes.

A deconstructable hydrophobic polymer network can store and release a biomolecule without altering or destroying the biomolecule. A deconstructable hydrophobic polymer network can store and release a biomolecule so that it can be further processed. Further processing of biomolecules can include sequencing reactions, amplification reactions, immunoprecipitation assays, spectrometry, or any preparatory steps thereof.

A hydrophobic polymer can be dissolved upon application of a polar or non-polar organic solvent.

A non-polar organic solvent is a solvent that has low dipole moment and can dissolve other non-polar substances. Non-limiting examples of non-polar organic solvents include benzene, toluene, hexanes, pentane, cyclohexane, octane, and dodecane.

A hydrophobic polymer can store and release a biomolecule without altering or destroying the biomolecule. A hydrophobic polymer can store and release a biomolecule so that it can be further processed. Further processing of biomolecules can include sequencing reactions, amplification reactions, immunoprecipitation assays, spectrometry, or any preparatory steps thereof.

### Methods for Processing a Biomolecule

A biomolecule (e.g., a sequence-controlled polymer or any other biomolecule as described herein) can be processed by a) contacting the sequence-controlled polymer with a DMAEMA-based polymer or an AETAC-based polymer in an aqueous solution (e.g., water) to form a first mixture; b) removing the aqueous solution from the first mixture; and c) contacting the first mixture with a hydrophobic agent to form a second mixture; wherein the second mixture comprises a hydrophobic polymer or polymer network that coats the biomolecule (e.g., a sequence-controlled polymer). A biomolecule (e.g., a sequence-controlled polymer) can be further processed by d) contacting the second mixture with a stimulus to form a third mixture and e) heating the third mixture, wherein heating the third mixture releases the biomolecule (e.g., a sequence-controlled polymer).

A biomolecule (e.g., a sequence-controlled polymer or any other biomolecule as described herein) can be processed by a) creating an emulsion of i) the sequence-controlled polymer in an aqueous solution (e.g., water) and ii) a DMAEMA-based polymer or an AETAC-based polymer in a hydrophobic agent; and b) applying a first stimulus to initiate the formation of a hydrophobic polymer or polymer network that coats the sequence-controlled polymer within the emulsion. A biomolecule (e.g., a sequence-controlled polymer) can be further processed by applying a second stimulus to the emulsion, thereby separating the hydrophobic agent from the emulsion and forming a hydrophobic polymer or polymer network that coats the biomolecule (e.g., a sequence-controlled polymer).

A DMAEMA-based polymer can be a DMAEMA-co-OEGMA-co-styrene polymer. An AETAC-based polymer can be an AETAC-co-OEGA-co-DDA polymer. Alternatively, an AETAC-based polymer can be an AETAC-co-PEGMA-co-DDA polymer.

A biomolecule can be processed using any of the hydrophobic agents as described herein (e.g., sytrene, DDA).

Methods for processing biomolecules can be performed at a temperature of at least about 5° C., at least about 10° C., at least about 15° C., at least about 20° C., at least about 25° C., at least about 30° C., at least about 35° C., at least about 40° C., at least about 45° C., at least about 50° C., at least about 55° C., at least about 60° C., at least about 65° C., at least about 70° C., at least about 75° C., at least about 80° C., at least about 85° C., at least about 90° C., at least about 95° C., at least about 100° C., at least about 105° C., at least about 110° C., at least about 115° C., at least about 120° C., at least about 125° C., at least about 130° C., at least about 135° C., at least about 140° C., at least about 145° C., or at least about 150° C. A hydrophobic polymer or polymer network can form at a temperature of at most about 150° C., at most about 145° C., at most about 140° C., at most about 135° C., at most about 130° C., at most about 125° C., at most about 120° C., at most about 115° C., at most about 110° C., at most about 105° C., at most about 100° C., at most about 95° C., at most about 90° C., at most about 85° C., at most about 80° C., at most about 75° C., at most about 70° C., at most about 65° C., at most about 60° C., at most about 55° C., at most about 50° C., at most about 45° C., at most about 40° C., at most about 35° C., at most about 30° C., at most about 25° C., at most about 20° C., at most about 15° C., at most about 10° C., or at most about 5° C.

Biomolecules can be barcoded or tagged prior to any of the processing methods described herein. Alternatively, biomolecules can be barcoded or tagged concurrently with any of the processing methods described herein. Alternatively, biomolecules can be barcoded or tagged after any of the processing methods described herein.

A biomolecule can be contacted with a stimulus during processing. A stimulus can be light, heat, and/or a deconstruction solution. A deconstruction solution can be any deconstruction solution as described herein. A stimulus (e.g., heat, light) used during thermoset formation can be the same stimulus used during the deconstruction of the thermoset. Alternatively, a stimulus used during thermoset formation can be a different stimulus as is used during deconstruction of the thermoset.

Processing a biomolecule for storage in a thermoset can occur in at least about 30 minutes, at least about 1 hour, at least about 2 hours, at least about 3 hours, at least about 4 hours, at least about 5 hours, at least about 6 hours, at least about 7 hours, at least about 8 hours, at least about 9 hours, or at least about 10 hours. Processing a biomolecule for storage in a thermoset can occur in at most about 10 hours, at most about 9 hours, at most about 8 hours, at most about 7 hours, at most about 6 hours, at most about 5 hours, at most about 4 hours, at most about 3 hours, at most about 2 hours, at most about 1 hour, or at most about 30 minutes.

Processing a biomolecule for release from a thermoset can occur in at least about 30 minutes, at least about 1 hour, at least about 2 hours, at least about 3 hours, at least about 4 hours, at least about 5 hours, at least about 6 hours, at least about 7 hours, at least about 8 hours, at least about 9 hours, or at least about 10 hours. Processing a biomolecule for release from a thermoset can occur in at most about 10 hours, at most about 9 hours, at most about 8 hours, at most about 7 hours, at most about 6 hours, at most about 5 hours, at most about 4 hours, at most about 3 hours, at most about 2 hours, at most about 1 hour, or at most about 30 minutes.

The total processing time for a method as described herein can occur in at least about 30 minutes, at least about 1 hour, at least about 2 hours, at least about 3 hours, at least about 4 hours, at least about 5 hours, at least about 6 hours, at least about 7 hours, at least about 8 hours, at least about 9 hours, or at least about 10 hours. The total processing time for a method as described herein can occur in at most about 10 hours, at most about 9 hours, at most about 8 hours, at most about 7 hours, at most about 6 hours, at most about 5 hours, at most about 4 hours, at most about 3 hours, at most about 2 hours, at most about 1 hour, or at most about 30 minutes.

A biomolecule (e.g., a sequence-controlled polymer or any other biomolecule as described herein) can be further processed by extracting the released sequence-controlled polymer from the third mixture. Extraction can comprise contacting the mixture with an organic solvent. Non-limiting examples of organic solvents can include ethyl acetate, chloroform, acetone, methanol, methylene chloride, ethanol, isopropyl alcohol, toluene, benzene, hexane, carbon tetrachloride, tetrahydrofuran, xylene, 1,2-dichloroethane, acetonitrile, acetic acid, diethyl ether, ethylene glycol, 1,4-dioxane, DMSO, DMF, 1-butanol, butanone, or chlorobenzene.

### Kits

A kit for storage and retrieval of a biomolecule (e.g., a sequence-controlled polymer) can comprise a flowcell comprising a support configured to bind the sequence-controlled polymer to the support; an amphiphilic polymer; a hydrophilic agent; and a hydrophobic agent.

A kit can comprise an amphiphilic polymer such as DMAEMA, AETAC, or any other amphiphilic polymer as described herein. A kit can comprise a hydrophilic agent such as OEGMA, OEGA, or any other hydrophilic agent as described herein. A kit can comprise a hydrophobic agent such as styrene, DDA, or any other hydrophobic agent as described herein.

A kit can comprise an aqueous solution (e.g., water). A kit can comprise a deconstruction solution. A deconstruction solution and/or its components can be aqueous solutions. A deconstruction solution can comprise a nucleophile (e.g., cysteamine or any other nucleophile as described herein), an organic base (e.g., DBU or any other organic base as described herein), and/or a polar solvent (e.g., DMF or any other polar solvent as described herein). A kit can comprise a deconstruction solution that is premixed. Alternatively, a kit can comprise the separately packaged components of a deconstruction solution.

A kit can comprise instructions for use.

### EXAMPLES

### Example 1: Encapsulation and Retrieval of Mus Musculus Genomic DNA in Deconstructable Thermoset Polymers

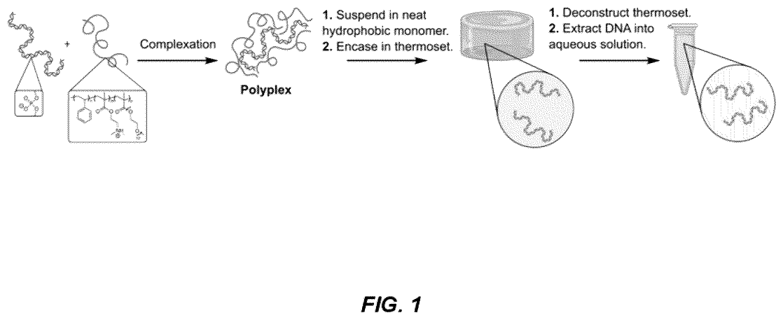

As exemplified in FIG. 1, a sequence-controlled polymer (e.g., DNA) was rendered compatible within the polymerization medium, which predominantly consists of hydrophobic solvents. The polymerization process was initiated using heat or light as a catalyst. The encapsulated DNA was liberated from its confinement and extracted into an aqueous solution for further analysis.

Polyplex assembly took place in dilute aqueous solutions at room temperature. Initially, aqueous stock solutions of DNA and the polyplex polymer were prepared at concentrations of 1 mg/mL and 10 mg/mL, respectively. The solutions were diluted in water within a 4 mL glass vial, resulting in a 1 mL polyplex solution with a DNA concentration of 0.2 mg/mL and 2-(dimethylamino)ethyl methacrylate. The solution was vortexed and incubated at room temperature for 24 hours to ensure complete assembly. The solution was lyophilized and resuspended in 1 mL of styrene to produce a styrene solution loaded with 0.2 mg/mL DNA.

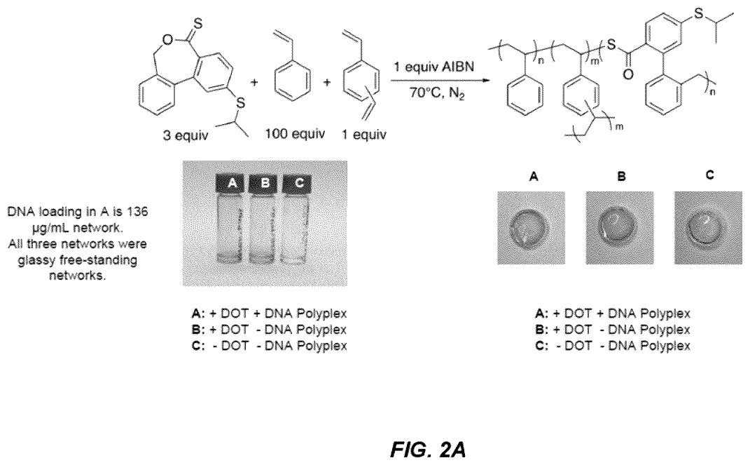

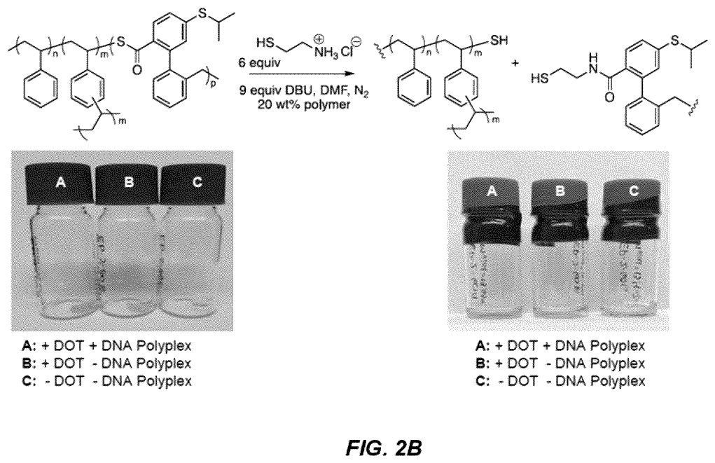

A glass vial was charged with divinylbenzene, azobisisobutyronitrile, the cleavable comonomer 2SiPr-DOT, and styrene. The styrene contained 0.2 mg/mL polyplexes to incorporate the DNA into the network. The vial was sealed with electrical tape and subjected to three cycles of freeze-pump-thaw for deoxygenation, followed by nitrogen backfilling. The vial was heated to 70° C. for 18 hours to facilitate complete network formation (FIG. 2A). Thermosets were extracted from the vials by gently striking the top of the glass vial with a hammer to shatter the vial and releasing the encapsulated material.

Deconstruction of thermosets with encapsulated DNA was achieved through treatment with cysteamine HCl and DBU to cleave the thioester bonds in the network's backbone. DNA extraction was conducted using liquid-liquid extraction with ethyl acetate and water (FIG. 2B).

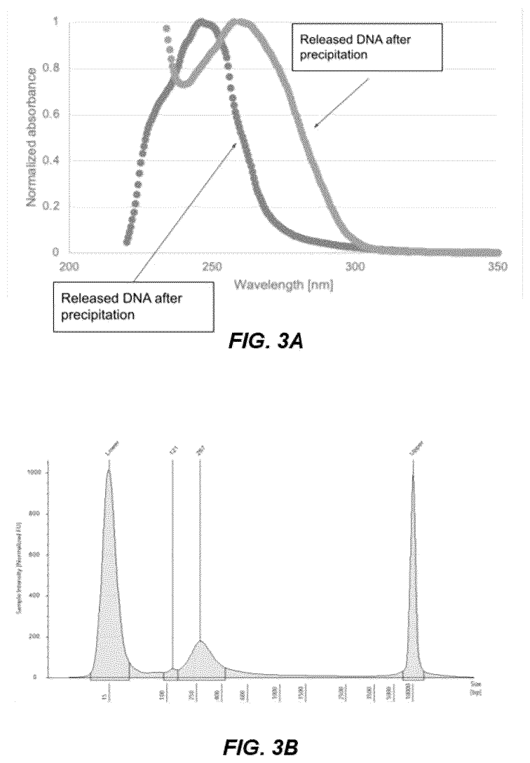

The released DNA exhibited the anticipated absorbance spectrum for DNA after ethanol precipitation with 3 M sodium acetate (FIG. 3A). The purified released DNA was then subjected to sequencing library preparation using enzymatic fragmentation, end repair, TruSeq adapter ligation, and dual-indexing via polymerase chain reaction. Fragment analysis of the indexed libraries yielded the expected sequencing library lengths (FIG. 3B). The fragment analysis demonstrated the compatibility and appropriateness of the extracted DNA for subsequent analytical processes, such as short-read DNA sequencing.

Example 2: Transferring 40mer ssDNA to Organic Solvents by Forming Polyplex in Emulsion

Dry, Hexachloro-Fluorescein (HEX)-labelled 40mer DNA (5′-HEX-ACTGACTGACTGACTGACTGACTGACTGACTGACTGACTG; SEQ ID NO: 1) was ordered from ThermoFisher. Stock solutions at 1-3 mg/mL were prepared by dissolving the dry DNA samples in Milli-Q water. Poly(AETAC-co-OEGA₄₈₀-co-DDA) was dissolved in various organic solvents (styrene, methyl methacrylate, n-butyl acrylate, tert-butyl acrylate, hexanes, toluene, ethyl acetate, and chloroform) at 10 mg/mL. The polymer solutions were combined with the aforementioned DNA solution at N/P=4. The nitrogen mass percent of each polymer was estimated based upon the incorporated amount of AETAC, as determined via ¹H NMR. Transfers were conducted in 1.5 mL Eppendorf tubes at 1,200 rpm and 40° C. After 10 minutes, the emulsion was centrifuged at 10,000 rpm for 2 minutes to separate the organic and aqueous layers.

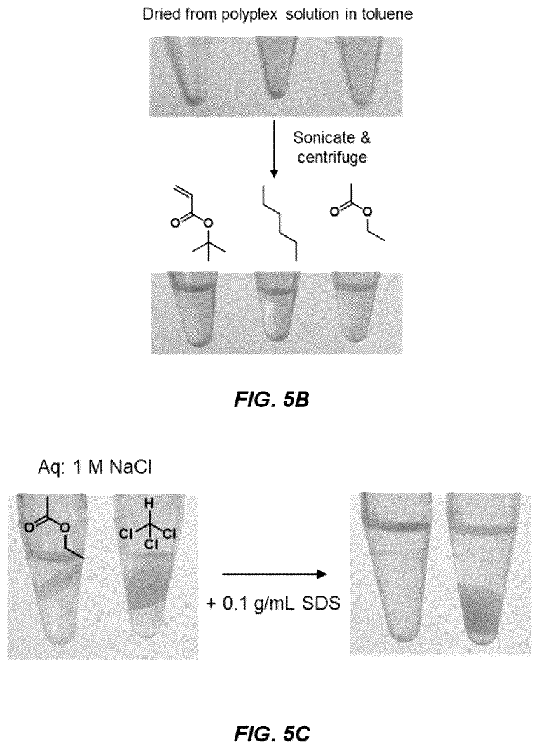

To extract polyplex-packaged DNA back to the aqueous phase, an aqueous solution of 1-2 M NaCl and 0.02-0.1 g/mL SDS was shaken with the organic solution to form an emulsion. HEX-labelled DNA were pink in color and the change in solution color indicated a successful transfer to styrene, methyl methacrylate, n-butyl acrylate, toluene, and chloroform (FIG. 5A). Centrifugation separated the two layers, and the pink aqueous layer was pipetted out.

The transfers were completed in 10 minutes for styrene, methyl methacrylate, n-butyl acrylate, toluene, and chloroform (FIG. 5A). For tert-butyl acrylate, hexanes, and ethyl acetate, the transfer was accomplished instead by resuspending a dried polyplex that was prepared from emulsion transfer using one of the aforementioned solvents (e.g., toluene) (FIG. 5B).

To re-extract polyplex-packaged DNA into aqueous phase, the addition of 5 M NaCl to the biphasic mixture was insufficient in breaking down the polycation-polyanion complexation, as the ethyl acetate and chloroform layers remained pink, respectively. However, upon the addition of 0.1 g/mL SDS, HEX-labelled DNA was successfully re-extracted back to the aqueous layer (FIG. 5C).

### Example 3: Encapsulation and Retrieval of 40mer ssDNA in Deconstructable Thermoset Polymers

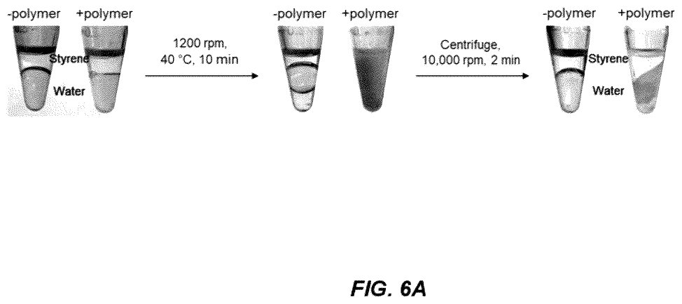

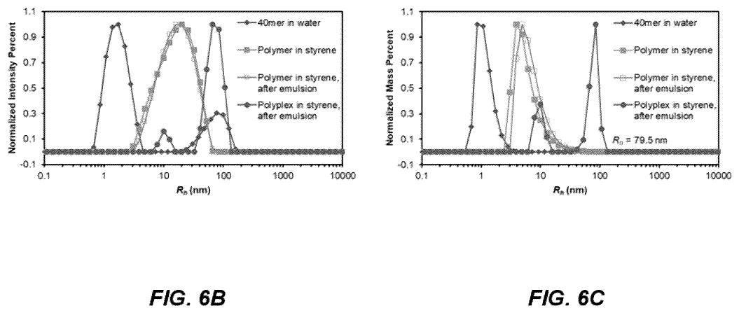

Dry, Hexachloro-Fluorescein (HEX)-labelled 40mer DNA (5′-HEX-ACTGACTGACTGACTGACTGACTGACTGACTGACTGACTG; SEQ ID NO: 2) was ordered from ThermoFisher. Stock solutions of 1-3 mg/mL were prepared by dissolving the dry DNA samples in Milli-Q water. Poly(AETAC-co-OEGA₄₈₀-co-DDA) was dissolved in inhibitor-free styrene at 10 mg/mL. The polymer solutions were combined with the aforementioned DNA solution at N/P=4. The nitrogen mass percent of each polymer was estimated based upon the incorporated mole ratio of AETAC, as determined via ¹H NMR. Transfers were conducted in 1.5 mL Eppendorf tubes at 1,200 rpm and 40° C. After 10 minutes, the emulsion was centrifuged at 10,000 rpm for 2 minutes to reveal a pink upper layer (styrene solution with 0.3-1 mg/mL Hex-labelled-DNA, packaged as a polyplex) and a colorless lower layer (water) (FIG. 6A). The pink styrene solution was pipetted out and can be used directly for bulk network formation.

A glass vial was charged with divinylbenzene, azobisisobutyronitrile, the cleavable comonomer 2SⁱPr-DOT, and styrene. The styrene contained 0.3-1 mg/mL polyplexes to incorporate DNA into the network. The vial was sealed with electrical tape and subjected to three cycles of freeze-pump-thaw for deoxygenation, followed by nitrogen backfilling. The vial was heated to 70° C. for 18 hours to facilitate complete network formation. Thermosets were extracted from the vials by gently striking the top of the glass vial with a hammer to shatter the vial and release the encapsulated material.

Deconstruction of thermosets with encapsulated DNA was achieved through treatment with cysteamine HCl and DBU to cleave the thioester bonds in the network's backbone. DNA extraction was conducted using liquid-liquid extraction with chloroform and water.

DLS characterization revealed the formation of styrene-soluble polyplexes with Rₕ˜80 nm (FIGS. 6B and 6C).

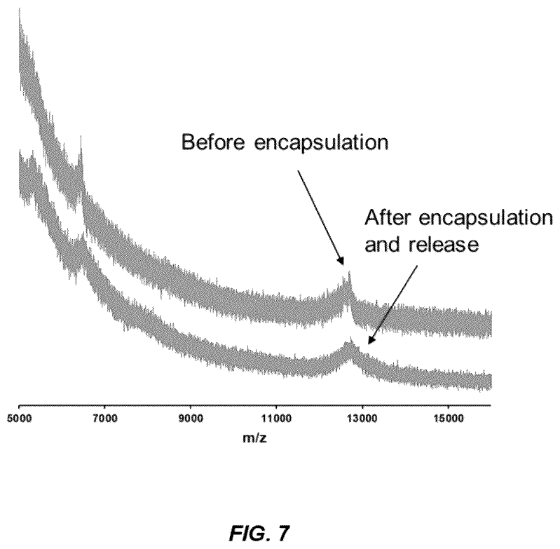

The released DNA exhibited the same m/z in MALDI-TOF MS characterization as the DNA sample before encapsulation (FIG. 7), indicating that the encasing and release process did not comprise the chemical integrity of DNA. The unchanged m/z illustrates that the integrity of DNA was preserved through the transfer, encapsulation, and release process.

### Example 4: Encapsulation and Retrieval of 40bp dsDNA in Deconstructable Thermoset Polymers

Dry, Hexachloro-Fluorescein (HEX)-labelled 40mer DNA (5′-HEX-AACATCTAATCTACATCTACCACTCACTATTACCATTCAC; SEQ ID NO: 3) and a complementary, unlabeled 40 mer (5′-GTGAATGGTAATAGTGAGTGGTAGATGTAGATTAGATGTT; SEQ ID NO: 4) were ordered from ThermoFisher. Stock solutions at 0.1-0.3 mg/mL were prepared by combining the two complementary strands in equimolar ratio in 0.1× PBS (pH 7.4), and the solution was annealed at room temperature for 1 hour. Poly(AETAC-co-OEGA₄₈₀-co-DDA) was dissolved in inhibitor-free styrene at 10 mg/mL. The polymer solutions were combined with the aforementioned DNA solution at N/P=4. The nitrogen mass percent of each polymer was estimated based upon the incorporated mole ratio of AETAC, as determined via ¹H NMR. Transfers were conducted in 1.5 mL Eppendorf tubes at 1,200 rpm and 40° C. After 60 minutes, the styrene solution was pipetted out and was used for bulk network formation.

A glass vial was charged with divinylbenzene, azobisisobutyronitrile, the cleavable comonomer 2SⁱPr-DOT, and styrene. The styrene contained 0.3-1 mg/mL polyplexes to incorporate DNA into the network. The vial was then sealed with electrical tape and subjected to three cycles of freeze-pump-thaw for deoxygenation, followed by nitrogen backfilling. The vial was heated to 70° C. for 18 hours to facilitate complete network formation. Thermosets were extracted from the vials by gently striking the top of the glass vial with a hammer to shatter the vial and release the encapsulated material.

Deconstruction of thermosets with encapsulated DNA was achieved through treatment with cysteamine HCl and DBU to cleave the thioester bonds in the network's backbone. DNA extraction was conducted using liquid-liquid extraction with chloroform and water.

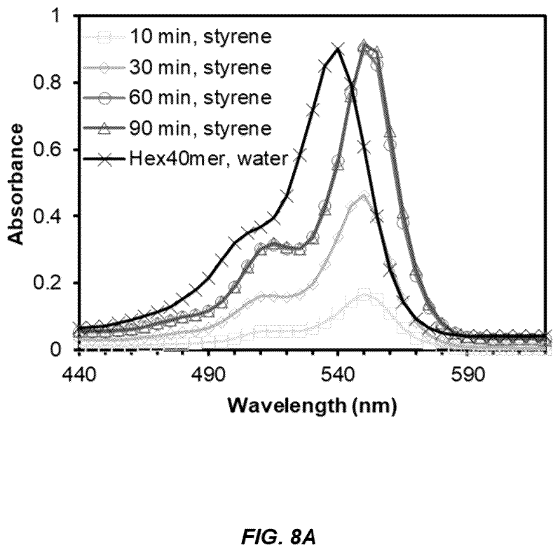

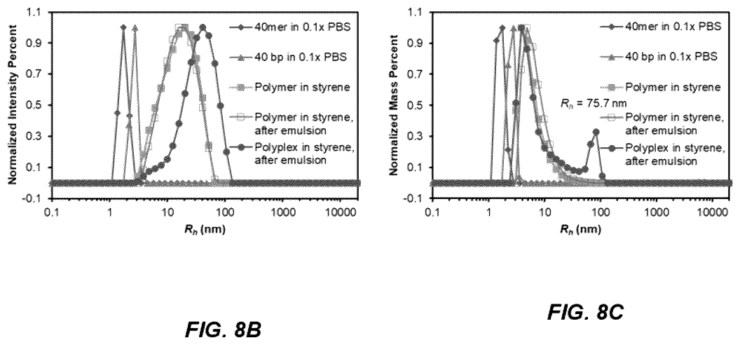

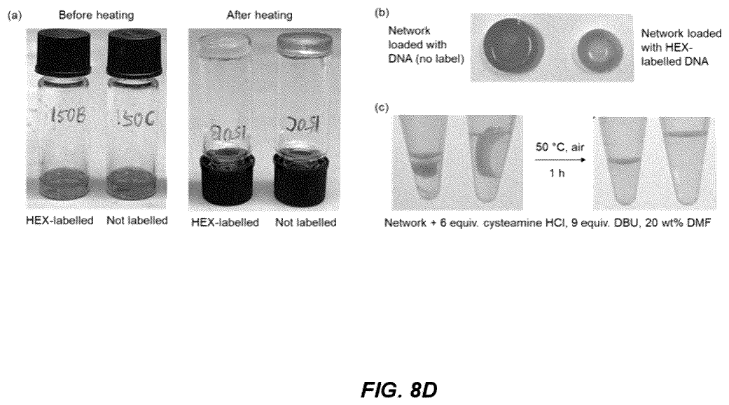

After different time points (10 min, 30 min, 60 min, and 90 min), the emulsion was centrifuged at 10,000 rpm for 2 minutes to separate the styrenic and aqueous layers, which were analyzed via UV-Vis absorbance spectroscopy to track the degree of DNA transfer (FIG. 8A), As evidenced by the unchanged absorbance level at 60 minutes and 90 minutes, the transfer was completed in 60 minutes.

DLS characterization revealed the formation of styrene-soluble polyplexes with Rₕ˜75 nm (FIGS. 8B-8C).

The styrene solution (loaded with HEX-labelled dsDNA) was incorporated into deconstructable thermoset, which was deconstructed completely within 1 h at 50° C. under air (FIG. 8D). The released and extracted DNA was quantified by measuring solution absorbance at 260 nm, which showed quantitative recovery.

### Example 5: Encapsulation and Retrieval of Human Genomic DNA in Deconstructable Thermoset Polymers

Human Genomic DNA (>50 kbp) (SKU: 11691112001) was purchased from Sigma Aldrich as 0.2 mg/mL solution in 10 mM Tris HCl, 1 mM EDTA, pH 8.0 and used as is. Poly(AETAC-co-OEGA₄₈₀-co-DDA) was dissolved in inhibitor-free styrene at 10 mg/mL. The polymer solutions were combined with the aforementioned DNA solution at N/P=4, 16, or 32. The nitrogen mass percent of each polymer was estimated based upon the incorporated mole ratio of AETAC, as determined via ¹H NMR. Transfers were conducted in 1.5 mL Eppendorf tubes at 1,200 rpm and 70° C. After 4 hours, the styrene solution was pipetted out and was used for bulk network formation.

A glass vial was charged with divinylbenzene, azobisisobutyronitrile, the cleavable comonomer 2SⁱPr-DOT, and styrene. The styrene contained 0.3-1 mg/mL polyplexes to incorporate DNA into the network. The vial was sealed with electrical tape and subjected to three cycles of freeze-pump-thaw for deoxygenation, followed by nitrogen backfilling. The vial was heated to 70° C. for 18 hours to facilitate complete network formation. Thermosets were extracted from the vials by gently striking the top of the glass vial with a hammer to shatter the vial and release the encapsulated material.

Deconstruction of thermosets with encapsulated DNA was achieved through treatment with cysteamine HCl and DBU to cleave the thioester bonds in the network's backbone. DNA extraction was conducted using liquid-liquid extraction with chloroform and water.

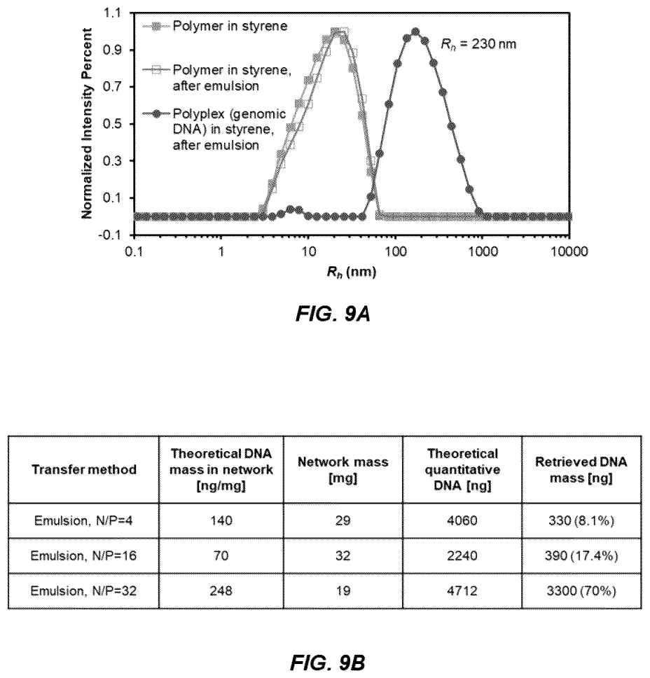

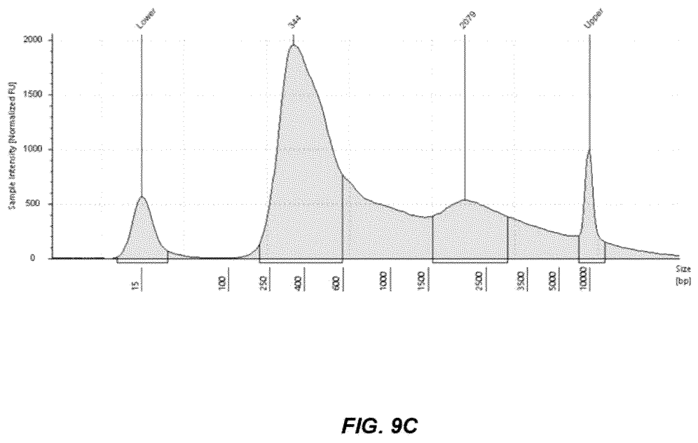

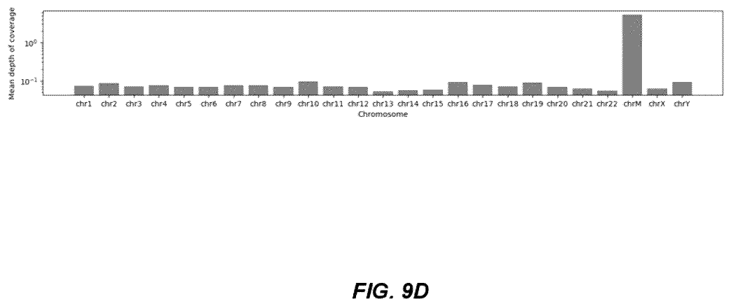

DLS characterization revealed the formation of styrene-soluble polyplexes with Rh ˜230 nm (FIG. 9A).

The released and extracted human genomic DNA was quantified by measuring solution absorbance at 260 nm, which shows 8-70% recovery (FIG. 9B). Higher N/P ratios enable more effective transfer of genomic DNA to styrene. The extracted DNA was sequenced (FIG. 9C). and the resulting sequencing data validated that the extracted DNA was mitochondrial DNA (FIG. 9D).

### Example 6: Additional Deconstructable Thermosets

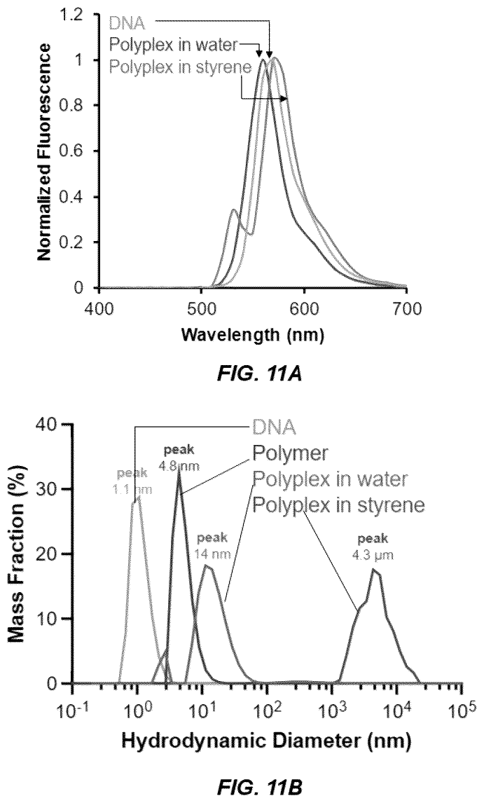

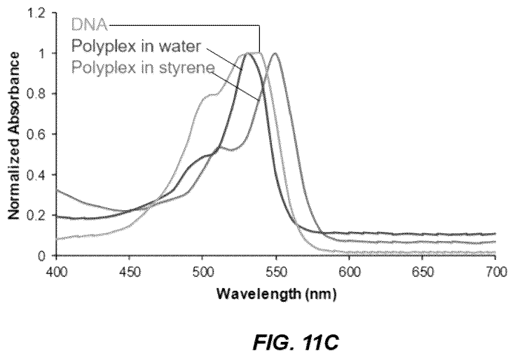

A p(OEGMA_{0.3}-co-DMAEMA_{0.3}-co-sytrene_{0.3}) DNA-polymer polyplex was formed by mixing dilute aqueous solutions of the polymer and DNA (C_{polymer}=0.5 mg/mL, P? N=1, C_{DNA}=136 μg/mL). Fluorescent DNA used was a 5′-HEX 40bp sequence (ACTGACTGACTGACTGACTGACTGACTGACTGACTGACTG; SEQ ID NO: 5). The mixture was incubated overnight at room temperature and polyplex formation was monitored by dynamic light scattering (DLS) (FIGS. 11A-11C).

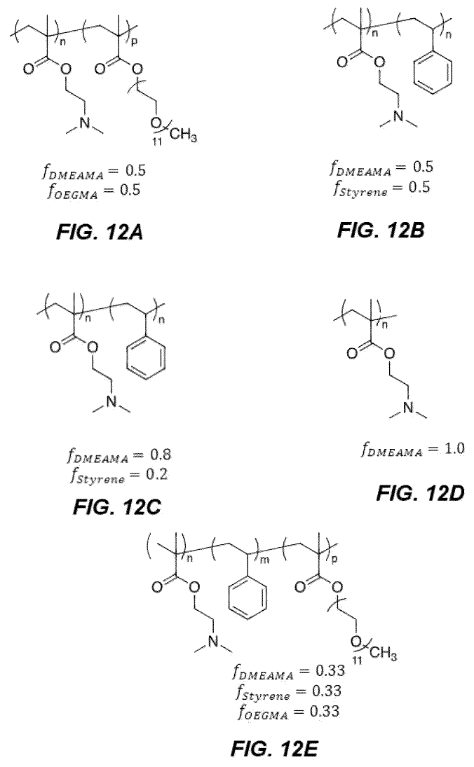

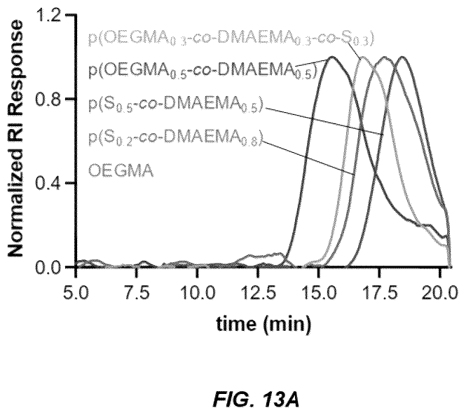

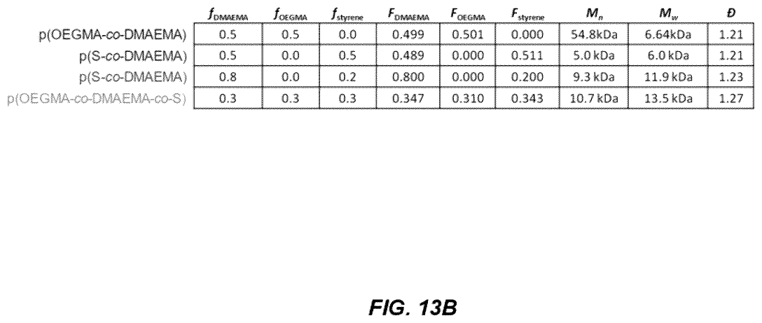

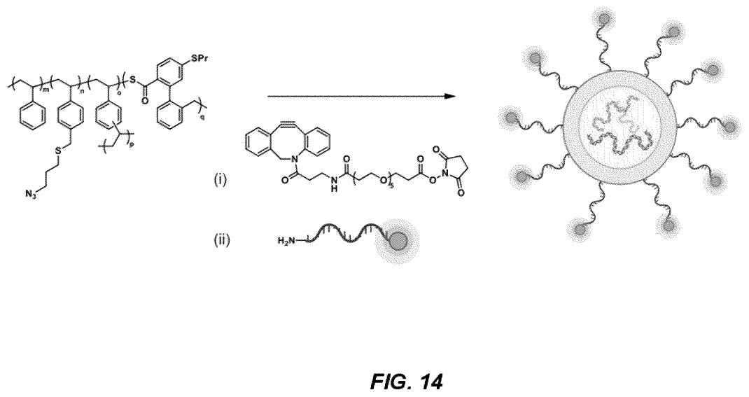

Thermosets were tested using p(DMAEMA-co-OEGMA) at a ratio of 1:1, p(DMAEMA-co-sytrene) at a ratio of 1:1; p(DMAEMA-co-sytrene) at a ratio of 1:4; p(DMAEMA-co-OEGMA-co-styrene) at a ratio of 1:1:1; and p(DMAEMA) (FIGS. 12A-12E). Thermoset formation was monitored by DLS (FIGS. 13A and 13B). Thermosets were tested for solubility in styrene, water, and 0.015M HCl.

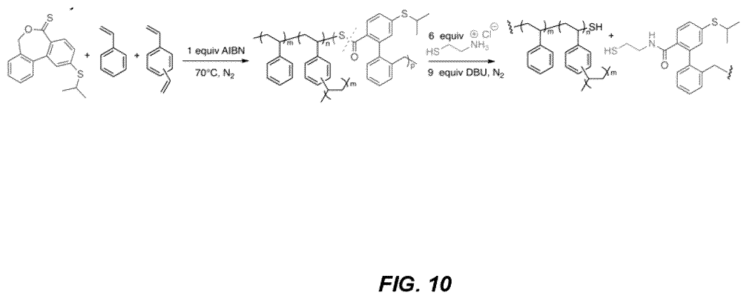

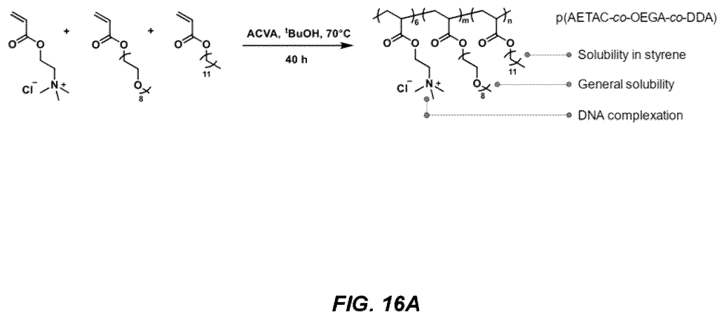

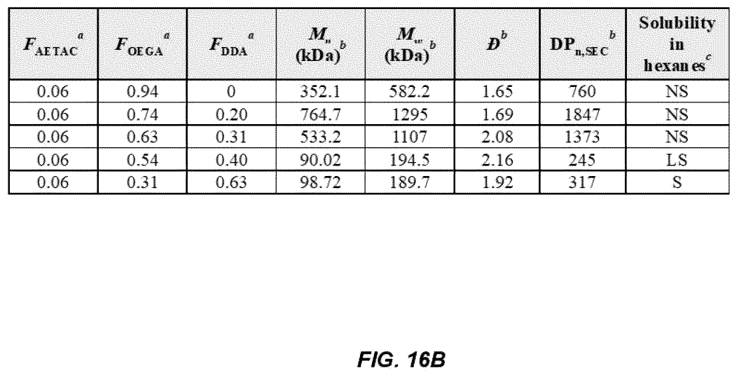

An additional thermoset was tested using p(AETAC-co-OEGA-co-DDA) (FIGS. 16A and 16B). Another thermoset was formed using poly(styrene-co-divinylbenzene), as shown in FIG. 10. The poly(styrene-co-divinylbenzene) thermoset was deconstructed using cysteamine HCl and DBU.

In some experiments, the thermosets were barcoded, as shown in FIG. 14.

### Example 7: Device for Thermoset Formation

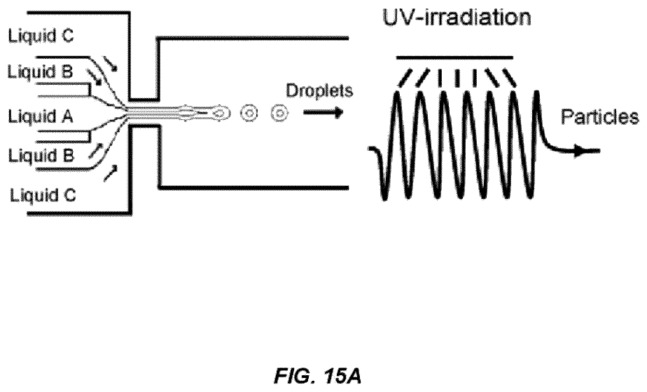

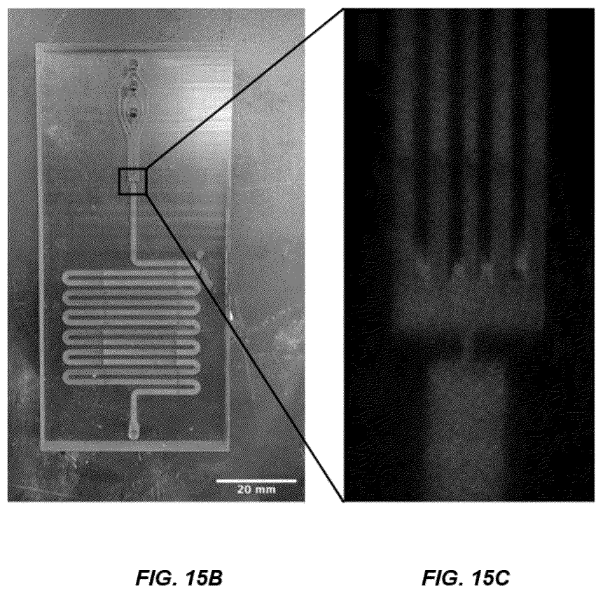

A BMF-HTL resin microfluidic flow-focusing device was created using 3D printing (FIGS. 15A-15C). The device comprised three liquid inlets. The three inlets flowed liquid into three separate channels which merged in order to synthesize the polyplex. The three channels merged at a junction where the liquids from each channel were allowed to combine into droplets. The droplets proceeded along a single channel while UV irradiation was added, allowing the emulsion of the three liquids to form into polyplex particles.

### Example 8: Deconstructable Polyplexes

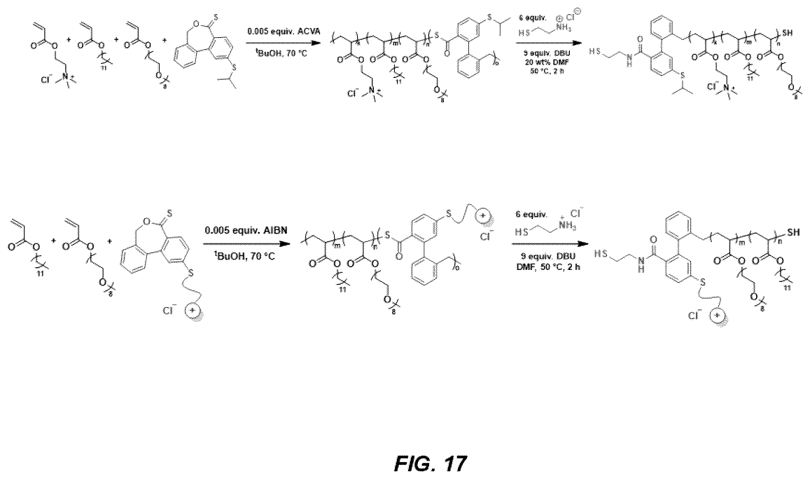

Deconstructable polycation-DNA complexes (polyplexes) were synthesized and evaluated. To make deconstructable polyplexes, polycations were furnished with a cleavable bond to render them degradable during polymer network deconstruction. In this example, the cleavable bond was installed in the form of a thioester bond, as shown in FIG. 17. The cationic moiety can be placed at the cleavable site, to facilitate complete breakdown of the polycations to monocationic fragments.

### Example 9: Using Polycations to Shuttle DNA Between Water and Hydrophobic Resin or Polymer Precursors

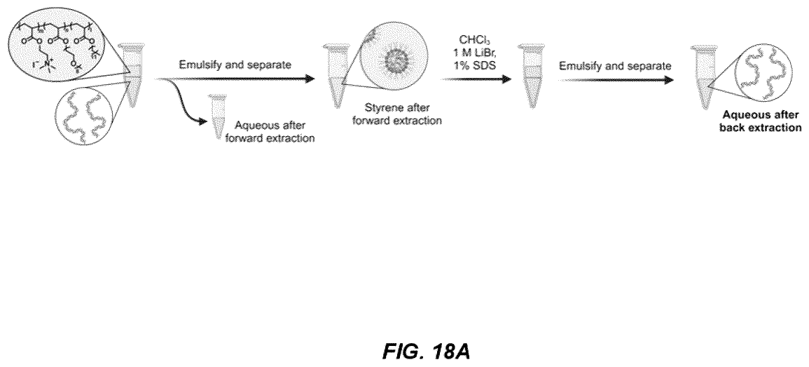

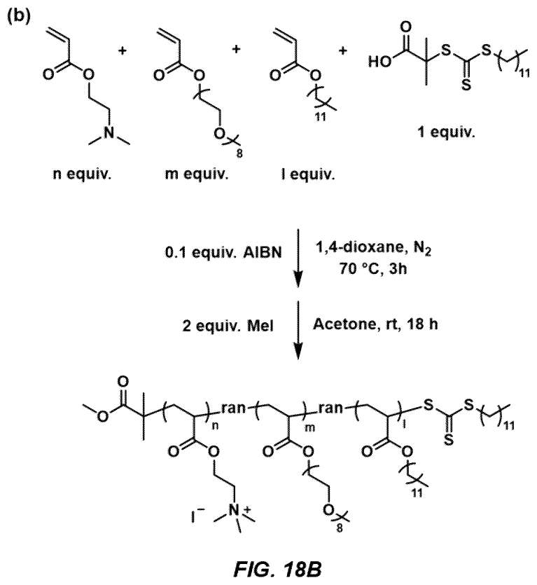

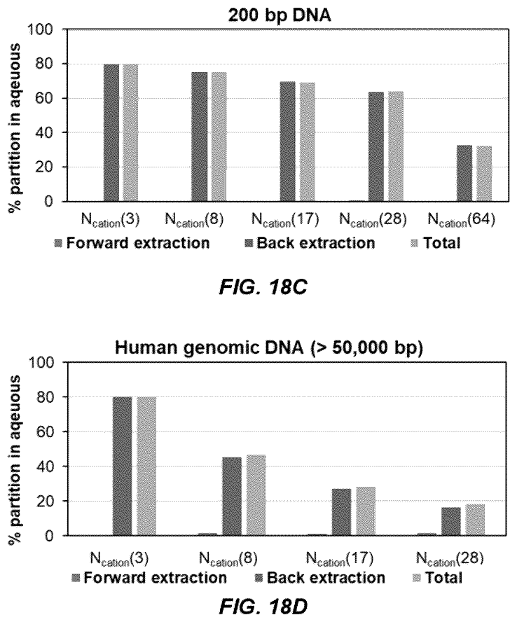

The performance of the polycations in reversibly shuttling DNA between water and styrene was evaluated, as shown in FIGS. 18A-D. FIG. 18A depicts the back-extraction of DNA from styrene to water through the action of anionic surfactant, sodium dodecyl sulfate (SDS), which breaks down the DNA-polycation complex. As shown in FIG. 18B, the molecular weight of polycations can be controlled during synthesis through controlled radical polymerization, such as Reversible Addition Fragmentation Chain Transfer (RAFT). The effect of the average number of cationic side chain on back-extraction efficiency was evaluated for both 200 bp DNA (FIG. 18C) and human genomic DNA (>50,000 bp) (FIG. 18D). The amount of dsDNA was quantified by measuring fluorescence enhancement due to dye intercalation, and the percentage partition in the aqueous layer was evaluated. The results showed that shorter polycations performed better in back-extraction experiments, especially for longer DNA.

### Example 10: Evaluating the Effect of Polycation-DNA Complexes on Polymer Network Formation and Polymer Network Properties

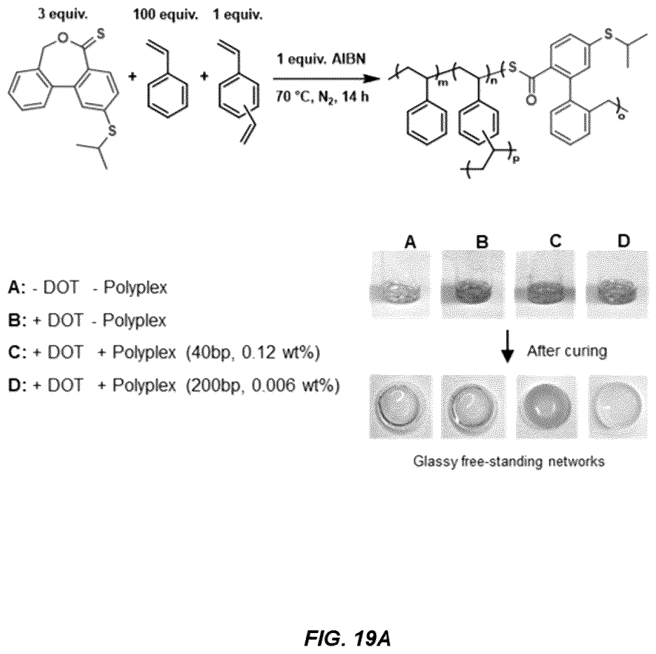

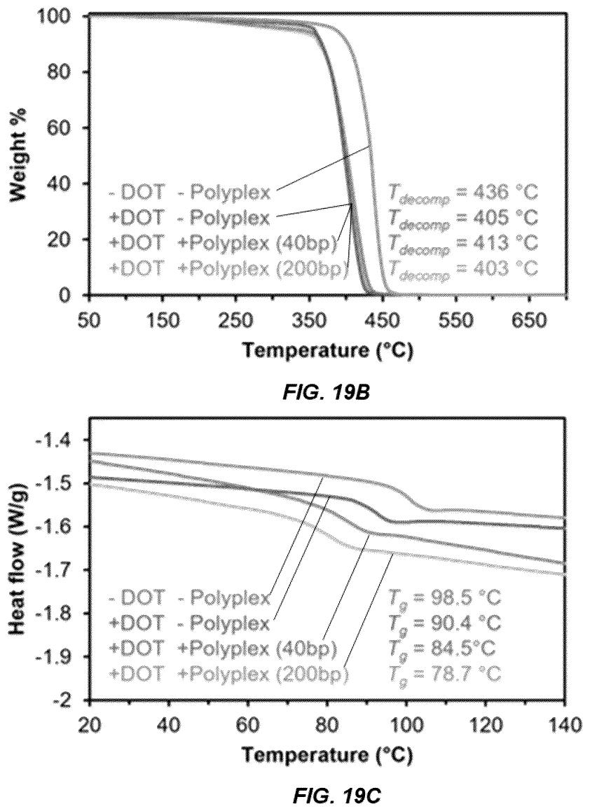

The polycation-DNA complex was further evaluated for its effect on i) the curing of styrene-divinylbenzene network (with or without DOT), ii) the decomposition temperature at inflection point (T_{decomp}), and iii) the glass transition temperature (T_{g}). The results showed that inclusion of polycation-DNA complex did not affect the curing of styrene-divinylbenzene network (with or without DOT) (as shown by the photos in FIG. 19A). While inclusion of DOT in the network sample decreased T_{decomp} by ˜30° C,, the polycation-DNA complex had little effect on T_{decomp} (FIG. 19B). As shown in FIG. 19C, inclusion of DOT lowered Tg by 8° C., while inclusion of the polycation-DNA complex only lowered the T_{g} by 6-12° C.

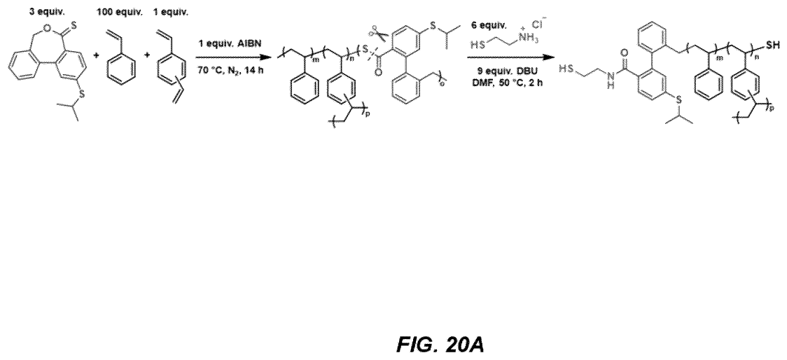

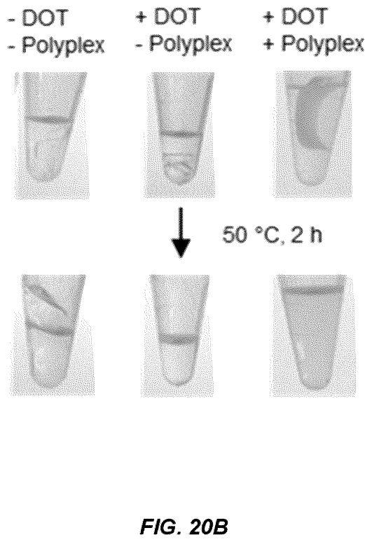

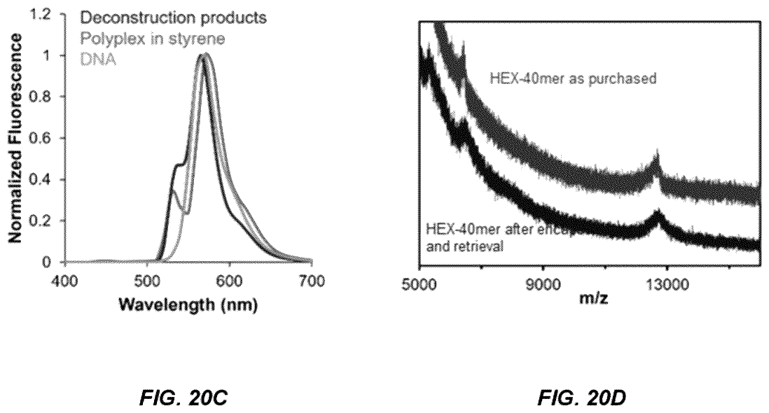

The synthesis and deconstruction of styrene-divinylbenzene network installed with thioester cleavable bonds was performed following the reaction scheme depicted in FIG. 20A. The inclusion of DOT inside the network was observed to be important for full deconstruction of the network, as shown in FIG. 20B. The deconstruction products were further characterized to evaluate the integrity of the DNA. Fluorescence spectra of the control HEX-labelled DNA, the polycation-DNA complex (polyplex), and the deconstruction products indicated that the polycation-DNA complex and the deconstruction products contained the same DNA (FIG. 20C). MALDI-TOF MS (matrix: DHAP, negative ion mode) characterization showed that the released DNA (following encapsulation and retrieval from the network) exhibited the same m/z as the DNA as purchased (5′HEX-AACATCTAATCTACATCTACCACTCACTATTACCATTCAC; SEQ ID NO: 3) before encapsulation (FIG. 20D), indicating that the DNA remained intact after polymerization and network deconstruction.

### Example 11: Encasing DNA in a Hydrophobic Polymer

DNA-polycation complex dissolved in styrene was added to water (water/styrene=10:1 (v/v)) containing a water-soluble initiator 2,2′-azobis[2-(2-imidazolin-2-yl)propane]dihydrochloride (AIBI) (0.001 to 0.01 equivalent with respect to styrene). After degassing the opaque emulsion by bubbling with nitrogen gas for 25 min, the emulsion was stirred and heated at 44° C. After 4 h, the reaction was removed from heat and stopped by the addition of a radical scavenger (butylated hydroxytoluene). This resulted in the formation of colloidally stable polystyrene particles that encapsulate DNA.

DNA-polycation complex dissolved in styrene (8-12 wt %) was added to an ethanol-water solution (70/30 wt/wt) containing AIBI (0.001 to 0.01 equivalent with respect to styrene) and poly(ethylene glycol) methacrylate (18 wt % with respect to styrene). After degassing the clear solution by bubbling with nitrogen gas for 25 min, the solution was stirred and heated at 44° C. Solution turned increasingly opaque throughout 4 h. After 4 h, the reaction was removed from heat and stopped by the addition of butylated hydroxytoluene. This resulted in the formation of colloidally stable polystyrene particles that encapsulate DNA.

DNA-polycation complex dissolved in styrene (8-12 wt %) was added to an ethanol-water solution (70/30 wt/wt) containing 2,2′-(disulfanediylbis(4,1-phenylene))diacetic acid (0.001 to 0.01 equivalent with respect to styrene) and poly(ethylene glycol) methacrylate (18 wt % with respect to styrene). After degassing the clear solution by bubbling with nitrogen gas for 25 min, the solution was stirred and irradiated using a 456 nm lamp (20-30 W, distance: 5 cm). The solution started turning opaque after 1 h. After 16 h, the reaction was removed from the light source and stopped by the addition of butylated hydroxytoluene. This resulted in the formation of colloidally stable polystyrene particles that encapsulate DNA.

DNA-polycation complex dissolved in styrene (8-12 wt %) was added to an ethanol-water solution (70/30 wt/wt) containing lithium phenyl-2,4,6-trimethylbenzoylphosphinate (0.001 to 0.01 equivalent with respect to styrene) and poly(ethylene glycol) methacrylate (18 wt % with respect to styrene). After degassing the clear solution by bubbling with nitrogen gas for 25 min, the solution was stirred and irradiated using a 456 nm lamp (20-30 W, distance: 5 cm). The solution started turning opaque after 30 min. After 5 h, the reaction was removed from the light source and stopped by the addition of butylated hydroxytoluene. This resulted in the formation of colloidally stable polystyrene particles that encapsulate DNA.

DNA encased in polystyrene particles was successfully retrieved by dissolving the polymer in a chloroform/ethanol solution (10/1 to 5/1 v/v) and extracting the DNA to an aqueous solution containing 1 M lithium bromide and 1 wt % sodium dodecyl sulfate. The amount of DNA recovered ranged from 5% to 90%, depending on polyplex formation method, polymerization condition, and DNA identity.

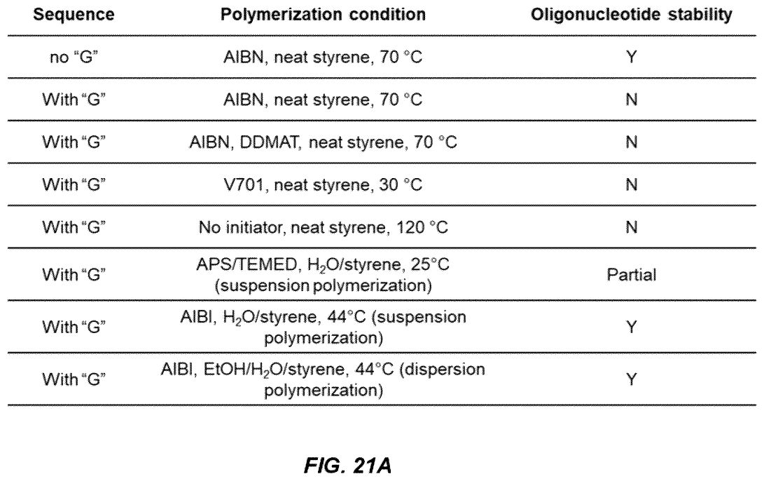

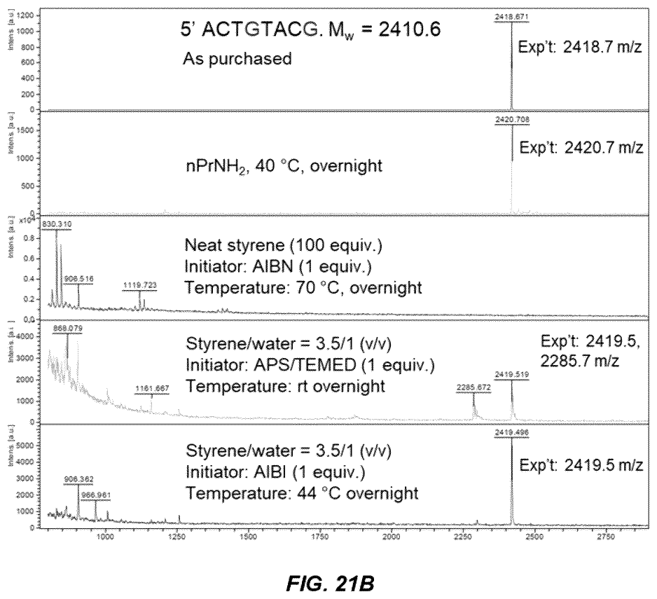

The stability of different oligonucleotides in various different styrene polymerization conditions was further evaluated by MALDI-TOF MS. FIG. 21A shows the polymerization conditions tested for an 8 or 40-base oligomer that either does not contain guanine (no “G”) or contains guanine (with “G”) and the corresponding oligonucleotide stability results as evaluated by MALDI-TOF MS. Example MALDI-TOF mass spectra of a 8-base oligomer (matrix: 2′,6′-Dihydroxyacetophenone, DHAP, negative ion mode) are depicted in FIG. 21B, showing oligonucleotide stability in various polymerization conditions. Here, the inclusion of guanine was tested since guanine has the lowest redox potential of all four nucleobases and is thus susceptible to oxidation. In the oligonucleotide stability results shown in FIG. 21A, “N” indicates no signal of intact DNA detected; “Y” indicates that intact DNA is detected, with no degraded products identified; and “Partial” indicates both intact DNA and degraded products were identified. Compared to suspension polymerization, dispersion polymerization (the last condition) yielded more homogenous DNA incorporation and particle size distribution. Other viable polymerization conditions tested include visible-light initiated photopolymerization, using a water-soluble photoinitiator.

While preferred embodiments of the present invention have been shown and described herein, it will be obvious to those skilled in the art that such embodiments are provided by way of example only. It is not intended that the invention be limited by the specific examples provided within the specification. While the invention has been described with reference to the aforementioned specification, the descriptions and illustrations of the embodiments herein are not meant to be construed in a limiting sense. Numerous variations, changes, and substitutions will now occur to those skilled in the art without departing from the invention. Furthermore, it shall be understood that all aspects of the invention are not limited to the specific depictions, configurations or relative proportions set forth herein which depend upon a variety of conditions and variables. It should be understood that various alternatives to the embodiments of the invention described herein may be employed in practicing the invention. It is therefore contemplated that the invention shall also cover any such alternatives, modifications, variations or equivalents. It is intended that the following claims define the scope of the invention and that methods and structures within the scope of these claims and their equivalents be covered thereby.

1.-155. (canceled)

156. A composition comprising:
a) [2-(acryloyloxy)ethyl]trimethylammonium chloride (AETAC),
b) oligo(ethylene glycol) methyl ether acrylate (OEGA), and
c) dodecyl acrylate (DDA).

157. The composition of claim 156, further comprising: d) 2-(Dodecylthiocarbonothioylthio)-2-methylpropionic acid (DDMAT).

158. A composition comprising:
a) an amphiphilic polymer that forms a complex with a sequence-controlled polymer;
b) a hydrophilic agent; and
c) a hydrophobic agent.

159. The composition of claim 158, further comprising: d) a chain-transfer agent.

160. The composition of claim 158, wherein the amphiphilic polymer comprises an amine-containing copolymer.

161. The composition of claim 160, wherein the amine-containing copolymer comprises a tertiary amine-containing polymer.

162. The composition of claim 158, wherein the amphiphilic polymer comprises a cation-containing copolymer.

163. The composition of claim 162, wherein the cation-containing copolymer comprises a quaternary amine-containing copolymer.

164. The composition of claim 158, wherein the hydrophilic agent comprises a hydrophilic monomer.

165. The composition of claim 158, wherein the hydrophobic agent comprises a hydrophobic monomer.

166. The composition of claim 158, further comprising a hydrophobic polymer or hydrophobic polymer network comprising a), b), and c).

167. The composition of claim 166, further comprising a non-aqueous solution comprising the hydrophobic polymer or hydrophobic polymer network.

168. The composition of claim 166, wherein the hydrophobic polymer or hydrophobic polymer network is stable for at least 1 day.

169. The composition of claim 166, wherein the hydrophobic polymer, hydrophobic polymer network, or the complex is deconstructable upon application of a stimulus.

170. The composition of claim 169, wherein the stimulus comprises light, heat, a deconstruction solution, or any combination thereof.

171. The composition of claim 158, wherein the complex further comprises a labile bond selected from a thioester bond, a disulfide bond, a carbamate bond, an amide bond, an acetal bond, an orthoester bond, a phosphoester bond, an anhydride bond, a hydrazone/Schiff base bond, an oxime bond, a glycosidic bond, an imine bond, and an acyl-hydrazone bond.

172. The composition of claim 166, further comprising a sequence-controlled polymer, wherein the composition coats the sequence-controlled polymer.

173. The composition of claim 172, wherein the sequence-controlled polymer is a biomolecule.

174. A method for processing a sequence-controlled polymer, comprising:
a) contacting the sequence-controlled polymer with a DMAEMA-based polymer or an AETAC-based polymer in an aqueous solution to form a first mixture;
b) removing the aqueous solution from the first mixture; and
c) contacting the first mixture with a hydrophobic agent to form a second mixture;
wherein the second mixture comprises a hydrophobic polymer or hydrophobic polymer network that coats the sequence-controlled polymer.
